(19) United States
(12) Patent Application Publication
(10) Pub. No.: US 2018/0092923 A1
(43) Pub. Date: Apr. 5, 2018

(54) METHODS FOR TREATING LYSOSOMAL STORAGE DISORDERS

(71) Applicant: BIOMARIN PHARMACEUTICAL INC., Novato, CA (US)

(72) Inventors: Leonard Post, Vacaville, CA (US); Brett E. Crawford, San Diego, CA (US); Shripad Bhagwat, San Diego, CA (US)

(21) Appl. No.: 15/566,950

(22) PCT Filed: Apr. 14, 2016
(86) PCT No.: PCT/US16/27473

§ 371 Date: Oct. 16, 2017

Related U.S. Application Data
(60) Provisional application No. 62/147,712, filed on Apr. 15, 2015.

(51) Int. Cl. A61K 31/5377; A61P 3/00

(52) U.S. Cl. CPC A61K 31/5377; A61P 3/00

(57) ABSTRACT

Described herein are methods using compounds of Formula (I), (Ia), and (Ib) to treat or prevent a lysosomal storage disorder, methods of making such compounds, and methods of using pharmaceutical compositions and medicaments containing such compounds.

### CROSS REFERENCE TO RELATED APPLICATIONS

This application claims the benefit of U.S. Provisional Application No. 62/147,712, filed on Apr. 15, 2015, which is incorporated herein by reference in its entirety.

### FIELD

Described herein are methods of using compounds to treat or prevent lysosomal storage disorders, such as an MPS disorder, methods of making such compounds, and methods of using pharmaceutical compositions and medicaments containing such compounds.

### BACKGROUND

In some lysosomal storage diseases, a person is missing a key enzyme (or has an enzyme which does not function normally) where the degradation of glycosylaminoglycans (GAGs) is impaired resulting in abnormal accumulation of these GAGs and disease. Substrate reduction therapy (SRT) offers an approach to treating diseases by inhibiting the formation of the substrate upon which the missing or abnormally-functioning enzyme acts. One potential therapeutic approach is to reduce the rate of biosynthesis of the GAG to offset the catabolic defect, restoring the balance between the rate of biosynthesis and the rate of catabolism. Small molecule inhibitors of GAG biosynthesis can reduce the amount of substrate formed and have the potential to treat diseases with CNS pathology.

Heparan sulfate (HS) is one such GAG found in mammals comprising glucosamine and uronic acid groups. In certain instances, heparan sulfate is bound to a core protein via a linkage tetrasacchraide, which generally has the structure -GlcAβGalβ3Galβ4Xylβ-O—. The cell surface of most mammalian cells contains membrane anchored heparan sulfate proteoglycans (HSPGs) which have important functions in cell adhesion processes (Biochimica et Biophysica Acta—Molecular Cell Research 2001, 1541(3), 135). In certain lysosomal storage disorders, the ability of the lysosome to degrade and turnover HS is impaired.

HSPGs have been shown to promote formation of amyloid structures typical in amyloid diseases (such as Alzheimer's disease, Parkinson's disease, type 2 diabetes, and chronic hemodialysis-related amyloidosis) including colocalization with the amyloid plaques and may impart stability to the amyloid fibrils (Journal of Biological Chemistry 2002, 277, 18421; J. Neuroscience 2011, 31(5), 1644; Proceedings of the National Academy of Sciences USA 2005, 102(18), 6473).

In some lysosomal storage diseases, such as Neimann-Pick disease type C, the subject exhibits a complete or partial deficiency of the sphingomyelinase enzyme, which causes an accumulation of cholesterol and/or glycosphingolipids in lysosomes of, e.g., neurons. Methods which would, e.g., prevent overproduction of the causative species or allow for clearance of the accumulated compounds would be useful for treatment of such diseases.

Lysosomal storage disorders include diseases such as cholesteryl ester storage disease, gangliosidosis (e.g. GM1 gangliosidosis), Neimann-Pick disease (e.g. Neimann-Pick type C), and an MPS disorder (e.g. MPS I, MPS II, MPS IIIA, or MPS IIIB).

The compounds disclosed herein are useful to treat lysosomal storage disorders.

### SUMMARY

Disclosed herein is a method for treating a lysosomal storage disorder in a subject in need thereof, the method comprising administering to the subject a compound, or a pharmaceutically acceptable salt thereof, according to Formula (I):

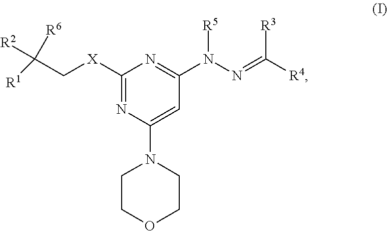

wherein
X is selected from the group consisting of: O, S, NH, and CH₂;
R¹ is selected from the group consisting of: C₁₋₃alkyl, —OR, oxazolidinonyl, morpholinyl, phenyl, pyridonyl, and pyridyl, wherein the C₁₋₃alkyl or phenyl is optionally substituted with 1-2 substituents independently selected from the group consisting of: —OCH₃ and —OH;
R is selected from H, C₁₋₃alkyl, phenyl, and pyridyl;
R² is selected from the group consisting of: H and CH₃; or
R¹ and R² taken together form a heterocyclic ring of 5-6 atoms optionally substituted with 1-2 C₁₋₃alkyl;
R³ is selected from the group consisting of: H and C₁₋₃alkyl;
R⁴ is selected from the group consisting of: aryl and heteroaryl, each of which is optionally substituted with C₁₋₃alkyl, halo, (C₁₋₃alkyl)-OH, —C(O)O(C₁₋₃alkyl), or —C(O)NH(C₁₋₃alkyl); or
R³ and R⁴ taken together form a carbocyclic, aryl, or heteroaryl ring, each of which is optionally substituted with 1-2 substituents independently selected from the group consisting of: halo, oxo, —OH, C₁₋₄alkyl, —O(C₁₋₄alkyl), and —NO₂;
R⁵ is selected from the group consisting of: H and CH₃;
R⁶ is selected from the group consisting of: H and CH₃; or
R² and R⁶ taken together form (═O) provided that R¹ and R² are not a heterocyclic ring.

Further provided herein is a method for treating a lysosomal storage disorder in a subject in need thereof, the method comprising administering to the subject a compound, or a pharmaceutically acceptable salt thereof, according to Formula (Ia):

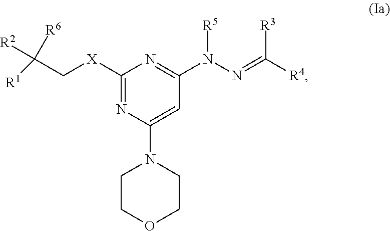

wherein
X is selected from the group consisting of: O, S, NH, and CH₂;
R¹ is selected from the group consisting of: C₁₋₃alkyl, —OR, morpholinyl, phenyl, pyridonyl, and pyridyl, wherein the C₁₋₃alkyl or phenyl is optionally substituted with 1-2 substituents independently selected from the group consisting of: —OCH₃ and —OH;
R is selected from H, C₁₋₃alkyl, phenyl, and pyridyl;
R² is selected from the group consisting of: H and CH₃; or
R¹ and R² taken together form a heterocyclic ring of 5-6 atoms optionally substituted with 1-2 C₁₋₃alkyl;
R³ is selected from the group consisting of: H and CH₃;
R⁴ is selected from the group consisting of: phenyl and indolyl, each of which is optionally substituted with C₁₋₃alkyl, halo, (C₁₋₃alkyl)-OH, —C(O)O(C₁₋₃alkyl), or —C(O)NH(C₁₋₃alkyl);
R⁵ is selected from the group consisting of: H and CH₃;
R⁶ is selected from the group consisting of: H and CH₃; or
R² and R⁶ taken together form (═O) provided that R¹ and R² are not a heterocyclic ring.

Also disclosed is a method for treating a lysosomal storage disorder in a subject in need thereof, the method comprising administering to the subject a compound, or a pharmaceutically acceptable salt thereof, according to Formula (Ib):

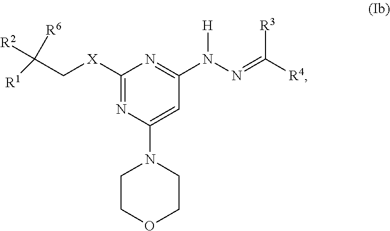

wherein
X is selected from the group consisting of: O and CH₂;
R¹ is selected from the group consisting of: C₁₋₃alkyl, oxazolidinonyl, and morpholinyl, wherein the C₁₋₃alkyl is optionally substituted with 1-2 substituents independently selected from the group consisting of: —OCH₃ and —OH;
R² is selected from the group consisting of: H and CH₃;
R³ and R⁴ taken together form a carbocyclic, aryl, or heteroaryl ring, each of which is optionally substituted with 1-2 substituents independently selected from the group consisting of: halo, oxo, —OH, C₁₋₄alkyl, —O(C₁₋₄alkyl), and —NO₂;
R⁶ is selected from the group consisting of: H and CH₃.

Further disclosed is a method for treating a lysosomal storage disorder in a subject in need thereof, the method comprising administering to the subject a compound, or a pharmaceutically acceptable salt thereof, having the structure:

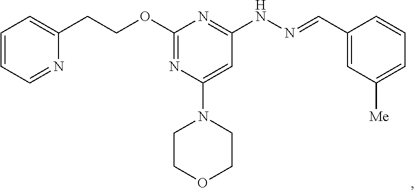 [SMILES: Cc1cccc(/C=N/Nc2cc(N3CCOCC3)nc(OCCc3ccccn3)n2)c1 ; ","]

Further provided herein is a method for treating Hunter syndrome in a subject in need thereof, the method comprising administering to the subject a compound, or a pharmaceutically acceptable salt thereof, having the structure:

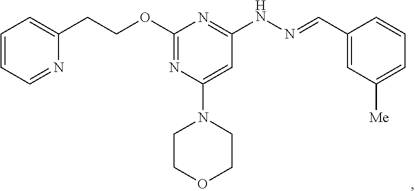 [SMILES: Cc1cccc(/C=N/Nc2cc(N3CCOCC3)nc(OCCc3ccccn3)n2)c1 ; ","]

### DETAILED DESCRIPTION

### Definitions

To facilitate understanding of the disclosure set forth herein, a number of terms are defined below. Generally, the nomenclature used herein and the laboratory procedures in organic chemistry, medicinal chemistry, and pharmacology described herein are those well-known and commonly employed in the art. Unless defined otherwise, all technical and scientific terms used herein generally have the same meaning as commonly understood by one of ordinary skill in the art to which this disclosure belongs.

As used throughout this application and the appended claims, the following terms have the following meanings:

The term “about” is meant to account for variations due to experimental error. All measurements reported herein are understood to be modified by the term “about”, whether or not the term is explicitly used, unless explicitly stated otherwise.

The term “acceptable” with respect to a formulation, composition or ingredient, as used herein, means having no persistent detrimental effect on the general health of the subject being treated.

The term “alkyl” refers to a straight or branched saturated hydrocarbon radical. Illustrative examples include, but are not limited to, methyl, ethyl, n-propyl, iso-propyl, n-butyl, sec-butyl, tert-butyl, n-pentyl, isopentyl, neopentyl, n-hexyl, 3-methylhexyl, 2,2-dimethylpentyl, 2,3-dimethylhexyl, n-heptyl, n-octyl, n-nonyl, and n-decyl. The term “C_{x-y}alkyl” refers to straight-chain alkyl and branched-chain alkyl groups that contain from x to y carbons in the chain.

The term “aryl” means a monovalent six- to eleven-membered, mono- or bi-carbocyclic ring, wherein the monocyclic ring is aromatic and at least one of the rings in the bicyclic ring is aromatic Representative examples include phenyl, naphthyl, tetrahydronaphthyl, dihydroindenyl, and indenyl, and the like.

The term “carbocyclyl” refers to a monocyclic or fused bicyclic, saturated or partially unsaturated (but not aromatic), hydrocarbon radical of three to ten carbon ring atoms. Fused bicyclic radical includes bridged ring systems. Carbocyclyl includes spirocycloalkyl rings. Unless stated otherwise, the valency of the group may be located on any atom of any ring within the radical, valency rules permitting. Representative examples of cyclic include but are not limited to:

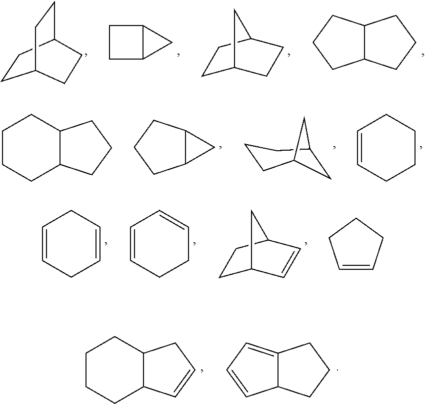

The term “heteroaryl” means a monocyclic or fused bicyclic radical of 5 to 10 ring atoms containing one, two, or three ring heteroatoms independently selected from —O—, —S(O)ₙ— (n is 0, 1, or 2), —N—, —N(H)—, and N-oxide, and the remaining ring atoms being carbon, wherein the ring comprising a monocyclic radical is aromatic and wherein at least one of the fused rings comprising a bicyclic radical is aromatic (but does not have to be a ring which contains a heteroatom, e.g. 2,3-dihydrobenzo[b][1,4]dioxin-6-yl). One or two ring carbon atoms of any nonaromatic rings comprising a bicyclic radical may be replaced by a —C(O)—, —C(S)—, or —C(═NH)— group. Fused bicyclic radical includes bridged ring systems. Unless stated otherwise, the valency may be located on any atom of any ring of the heteroaryl group, valency rules permitting. Heteroaryl includes, but is not limited to, chroman, thiochroman, triazolyl, tetrazolyl, pyrrolyl, imidazolyl, thienyl, furanyl, pyrazolyl, oxazolyl, isooxazolyl, oxadiazolyl, thiadiazolyl, indolyl, 2,3-dihydro-1H-indolyl (including, for example, 2,3-dihydro-1H-indol-2-yl or 2,3-dihydro-1H-indol-5-yl, and the like), indazolyl, phthalimidyl, benzimidazolyl, benzoxazolyl, benzofuranyl, benzothienyl, benzopyranyl, benzothiazolyl, pyridinyl, pyrazinyl, pyrimidinyl, pyridazinyl, quinolinyl, isoquinolinyl, tetrahydroisoquinolinyl (including, for example, tetrahydroisoquinolin-4-yl or tetrahydroisoquinolin-6-yl, and the like), pyrrolo[3,2-c]pyridinyl (including, for example, pyrrolo[3,2-c]pyridin-2-yl or pyrrolo[3,2-c]pyridin-7-yl, and the like), pyrrolo[1,2-b]pyridazinyl, imidazo[1,2-a]pyridinyl, thiazolyl, benzo[d][1,3]dioxolyl, 2,3-dihydrobenzo[b][1,4]dioxinyl, furo[2,3-d]thiazolyl, thieno[2,3-d]oxazolyl, thieno[3,2-b]furanyl, furo[2,3-d]pyrimidinyl, furo[3,2-b]pyridinyl, furo[3,2-c]pyridinyl, 6,7-dihydro-5H-cyclopenta[b]pyridinyl, and 7,8-dihydro-6H-cyclopenta[g]quinoxalinyl; and derivatives, N-oxide and protected derivatives thereof.

“Heterocyclyl” means a saturated or partially unsaturated (but not aromatic) monovalent monocyclic group of 3 to 9 ring atoms or a saturated or partially unsaturated (but not aromatic) monovalent fused bicyclic group of 8 to 12 ring atoms in which one, two, or three ring heteroatoms are independently selected from —O—, —S(O)ₙ— (n is 0, 1, or

2), —N═, —NH—, and N-oxide, the remaining ring atoms being carbon. Fused bicyclic radical includes bridged ring systems. Unless otherwise stated, the valency of the group may be located on any atom of any ring within the radical, valency rules permitting. More specifically the term heterocyclyl includes, but is not limited to, azetidinyl, pyrrolidinyl, 2-oxopyrrolidinyl, oxazolidinonyl, 2,5-dihydro-1H-pyrrolinyl, 2,5-dioxo-1H-pyrrolyl, 2,5-dioxo-pyrrolidinyl, 2,5-dihydro-1H-pyrrolyl, piperidinyl, 2-oxopiperidinyl, 4-piperidonyl, morpholinyl, piperazinyl, 2-oxopiperazinyl, dioxopiperazinyl, pyranyl, tetrahydropyranyl, tetrahydrothiopyranyl, 1,3-dioxinyl, 1,3-dioxanyl, 1,4-dioxinyl, 1,4-dioxanyl, 1,3-dioxolanyl, thiomorpholinyl, thiamorpholinyl, perhydroazepinyl, pyrazolidinyl, imidazolinyl, imidazolidinyl, 2,4-dioxo-imidazolidinyl, dihydropyridinyl, tetrahydropyridinyl, oxazolinyl, oxazolidinyl, oxazolidinonyl, isoxazolidinyl, thiazolinyl, thiazolidinyl, quinuclidinyl, isothiazolidinyl, octahydroindolyl, octahydroisoindolyl, decahydroisoquinolyl, tetrahydrofuryl, tetrahydropyranyl, 2-azaspiro[3.3]heptanyl, 7-azabicyclo[2.2.1]heptanyl, and 8-azabicyclo[3.2.1]octanyl, and the derivatives thereof and N-oxide (for example 1-oxido-pyrrolidin-1-yl) or a protected derivative thereof.

The term “stereoisomers” includes (but are not limited to) geometric isomers, enantiomers, diastereomers, and mixtures of geometric isomers, enantiomers or diastereomers. In some embodiments, individual stereoisomers of compounds are prepared synthetically from commercially available starting materials which contain asymmetric or chiral centers or by preparation of racemic mixtures followed by resolution. These methods of resolution are exemplified by (1) attachment of a mixture of enantiomers to a chiral auxiliary, separation of the resulting mixture of diastereomers by recrystallization or chromatography and liberation of the optically pure product from the auxiliary or (2) direct separation of the mixture of optical enantiomers on chiral chromatographic column.

Compounds in this application were drawn using ChemDraw Ultra version 12.0 (PerkinElmer, Inc.) The chemical names were generated from the drawn structures using ChemDraw Ultra ver. 12.0.

As used herein, “amelioration” of the symptoms of a particular disorder by administration of a particular compound or pharmaceutical composition refers to any lessening of severity, delay in onset, slowing of progression, or shortening of duration, whether permanent or temporary, lasting or transient that can be attributed to or associated with administration of the compound or composition.

The term “carrier” includes pharmaceutically inert, inorganic or organic carriers for the production of pharmaceutical compositions. Lactose, corn starch, or derivatives thereof, talc, stearic acid or its salts and the like can be used, for example, as such carriers for tablets, coated tablets, dragees and hard gelatin capsules. Suitable carriers for soft gelatin capsules are, for example, vegetable oils, waxes, fats, semi-solid and liquid polyols and the like. Depending on the nature of the active ingredient no carriers are, however, usually required in the case of soft gelatin capsules, other than the soft gelatin itself. Suitable carriers for the production of solutions and syrups are, for example, water, polyols, glycerol, vegetable oils and the like. Suitable carriers for suppositories are, for example, natural or hardened oils, waxes, fats, semi-liquid or liquid polyols and the like.

The term “diluent” refers to chemical compounds that are used to dilute the compound of interest prior to delivery. Diluents include chemicals used to stabilize compounds because they provide a more stable environment. Salts dissolved in buffered solutions (which also can provide pH control or maintenance) are utilized as diluents in some or any embodiments, including, but not limited to a phosphate buffered saline solution.

The terms “effective amount” or “therapeutically effective amount,” as used herein, refer to a sufficient amount of an agent or a compound being administered which will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is determined using any suitable technique, such as a dose escalation study.

The term “excipient” or “pharmaceutically acceptable excipient” means a pharmaceutically-acceptable material, composition, or vehicle, such as a liquid or solid filler, diluent, carrier, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Remington: The Science and Practice of Pharmacy, 21st ed.; Lippincott Williams & Wilkins: Philadelphia, Pa., 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, Fla., 2009.

The term “pharmaceutically acceptable salt” refers to a formulation of a compound that does not cause significant irritation to an organism to which it is administered and does not abrogate the biological activity and properties of the compound. In certain instances, pharmaceutically acceptable salts are obtained by reacting a compound described herein, with acids such as hydrochloric acid, hydrobromic acid, sulfuric acid, nitric acid, phosphoric acid, methanesulfonic acid, ethanesulfonic acid, p-toluenesulfonic acid, salicylic acid and the like. In some instances, pharmaceutically acceptable salts are obtained by reacting a compound having acidic group described herein with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like, or by other methods previously determined. The pharmacologically acceptable salt s not specifically limited as far as it can be used in medicaments. Examples of a salt that the compounds described hereinform with a base include the following: salts thereof with inorganic bases such as sodium, potassium, magnesium, calcium, and aluminum; salts thereof with organic bases such as methylamine, ethylamine and ethanolamine; salts thereof with basic amino acids such as lysine and omithine; and ammonium salt. The salts may be acid addition salts, which are specifically exemplified by acid addition salts with the following: mineral acids such as hydrochloric acid, hydrobromic acid, hydroiodic acid, sulfuric acid, nitric acid, and phosphoric acid:organic acids such as formic acid, acetic acid, propionic acid, oxalic acid, malonic acid, succinic acid, fumaric acid, maleic acid, lactic acid, malic acid, tartaric acid, citric acid, methanesulfonic acid, and ethanesulfonic acid; acidic amino acids such as aspartic acid and glutamic acid.

The term “pharmaceutical composition” refers to a mixture of a compound described herein with other chemical components, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to: intravenous, oral, aerosol, parenteral, ophthalmic, pulmonary and topical administration.

The term “subject” refers to an animal, including, but not limited to, a primate (e.g., human), monkey, cow, pig, sheep, goat, horse, dog, cat, rabbit, rat, or mouse. The terms “subject” and “patient” are used interchangeably herein in reference, for example, to a mammalian subject, such as a human.

The terms “treat,” “treating,” and “treatment,” in the context of treating a disease or disorder, are meant to include alleviating or abrogating a disorder, disease, or condition, or one or more of the symptoms associated with the disorder, disease, or condition; or to slowing the progression, spread or worsening of a disease, disorder or condition or of one or more symptoms thereof. Often, the beneficial effects that a subject derives from a therapeutic agent do not result in a complete cure of the disease, disorder or condition.

### Methods of Use

Disclosed in this application are methods for treating a lysosomal storage disorder in a subject in need thereof, comprising administering a compound, or pharmaceutically acceptable salt thereof, according to Formula (I):

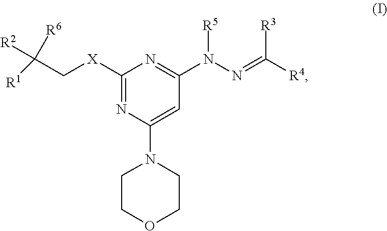

wherein
X is selected from the group consisting of: O, S, NH, and CH₂;
R¹ is selected from the group consisting of: C₁₋₃alkyl, —OR, oxazolidinonyl, morpholinyl, phenyl, pyridonyl, and pyridyl, wherein the C₁₋₃alkyl or phenyl is optionally substituted with 1-2 substituents independently selected from the group consisting of: —OCH₃ and —OH;
R is selected from H, C₁₋₃alkyl, phenyl, and pyridyl;
R² is selected from the group consisting of: H and CH₃; or
R¹ and R² taken together form a heterocyclic ring of 5-6 atoms optionally substituted with 1-2 C₁₋₃alkyl;
R³ is selected from the group consisting of: H and C₁₋₃alkyl;
R⁴ is selected from the group consisting of: aryl and heteroaryl, optionally substituted with C₁₋₃alkyl, halo, (C₁₋₃alkyl)-OH, —C(O)O(C₁₋₃alkyl), or —C(O)NH(C₁₋₃alkyl); or
R³ and R⁴ taken together form a carbocyclic, aryl, or heteroaryl ring, each of which is optionally substituted with 1-2 substituents independently selected from the group consisting of: halo, oxo, —OH, C₁₋₄alkyl, —O(C₁₋₄alkyl), and —NO₂;
R⁵ is selected from the group consisting of: H and CH₃;
R⁶ is selected from the group consisting of: H and CH₃; or
R² and R⁶ taken together form (═O) provided that R¹ and R² are not a heterocyclic ring.

In some embodiments, X is selected from the group consisting of: O and CH₂. For example, X can be O.

In some embodiments, R¹ is selected from the group consisting of: oxazolidinonyl, morpholinyl, pyridonyl, pyridyl, and —O-pyridyl. In some embodiments, R¹ is selected from the group consisting of: morpholinyl and pyridyl. For example, R¹ can be 2-pyridyl.

In some embodiments, R² is H.

In some embodiments, R³ is H.

In some embodiments, R⁴ is a heteroaryl optionally substituted with C₁₋₃alkyl. For example, R⁴ can be indolyl. In some embodiments, R⁴ is an aryl optionally substituted with C₁₋₃alkyl, halo, (C₁₋₃alkyl)-OH, —C(O)O(C₁₋₃alkyl), or —C(O)NH(C₁₋₃alkyl). In some embodiments, R⁴ is a phenyl substituted with C₁₋₃alkyl, halo, (C₁₋₃alkyl)-OH, —C(O)O(C₁₋₃alkyl), or —C(O)NH(C₁₋₃alkyl). For example, R⁴ can be 3-methylphenyl.

In some embodiments, R³ and R⁴ taken together form a heteroaryl optionally substituted with 1-2 substituents independently selected from the group consisting of: halo, oxo, —OH, C₁₋₄alkyl, and —O(C₁₋₄alkyl). In some embodiments, R³ and R⁴ taken together form an aryl optionally substituted with 1-2 substituents independently selected from the group consisting of: halo, —OH, C₁₋₄alkyl, —O(C₁₋₄alkyl), and —NO₂.

In some embodiments, R⁵ is H.

In some embodiments, R⁶ is H.

In some embodiments, R², R⁵, and R⁶ are H.

In some embodiments, R², R³, R⁵, and R⁶ are H.

Also disclosed are methods for treating a lysosomal storage disorder in a subject in need thereof, comprising administering a compound, or pharmaceutically acceptable salt thereof, according to Formula (Ia):

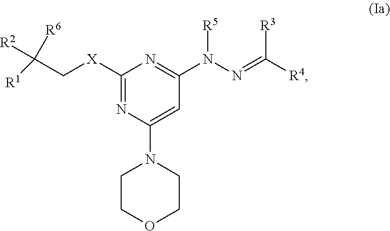

wherein
X is selected from the group consisting of: O, S, NH, and CH₂;
R¹ is selected from the group consisting of: C₁₋₃alkyl, —OR, morpholinyl, phenyl, pyridonyl, and pyridyl, wherein the C₁₋₃alkyl or phenyl is optionally substituted with 1-2 substituents independently selected from the group consisting of: —OCH₃ and —OH;
R is selected from H, C₁₋₃alkyl, phenyl, and pyridyl;
R² is selected from the group consisting of: H and CH₃; or
R¹ and R² taken together form a heterocyclic ring of 5-6 atoms optionally substituted with 1-2 C₁₋₃alkyl;
R³ is selected from the group consisting of: H and CH₃;
R⁴ is selected from the group consisting of: phenyl and indole, each of which is optionally substituted with C₁₋₃alkyl, halo, (C₁₋₃alkyl)-OH, —C(O)O(C₁₋₃alkyl), or —C(O)NH(C₁₋₃alkyl);
R⁵ is selected from the group consisting of: H and CH₃;
R⁶ is selected from the group consisting of: H and CH₃; or
R² and R⁶ taken together form (═O) provided that R¹ and R² are not a heterocyclic ring.

In some embodiments, X is selected from the group consisting of: O and CH₂. For example, X can be O.

In some embodiments, R¹ is selected from the group consisting of: —O-pyridyl, morpholinyl, pyridonyl, and pyridyl. For example, R¹ can be 2-pyridyl.

In some embodiments, R² is H.

In some embodiments, R³ is H.

In some embodiments, R⁴ is phenyl optionally substituted with C₁₋₃alkyl, halo, (C₁₋₃alkyl)-OH, —C(O)O(C₁₋₃alkyl), or —C(O)NH(C₁₋₃alkyl). For example, R⁴ can be 3-halophenyl. In some embodiments, R⁴ can be 3-methylphenyl.

In some embodiments, R⁵ is H.

In some embodiments, R⁶ is H.

In some embodiments, R², R⁵, and R⁶ are H.

In some embodiments, R², R³, R⁵, and R⁶ are H.

In some embodiments, the compound or pharmaceutically acceptable salt of Formula (Ia) is selected from:

| Cmpd | | |
|---|---|---|
| # | Structure | Chemical Name |
| 1 | 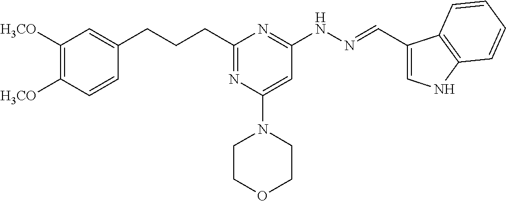 [SMILES: COc1ccc(CCCc2nc(N/N=C/c3c[nH]c4ccccc34)cc(N3CCOCC3)n2)cc1OC] | (E)-4-(6-(2-((1H-indol-3- yl)methylene)hydrazinyl)-2-(3-(3,4- dimethoxyphenyl)-propyl)-pyrimidin-4- yl)morpholine |
| 2 | 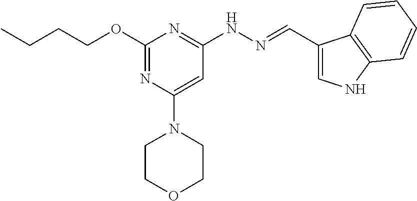 [SMILES: CCCCOc1nc(N/N=C/c2c[nH]c3ccccc23)cc(N2CCOCC2)n1] | (E)-4-(6-(2-((1H-indol-3- yl)methylene)hydrazinyl)-2- butoxypyrimidin-4-yl)morpholine |
| 3 | 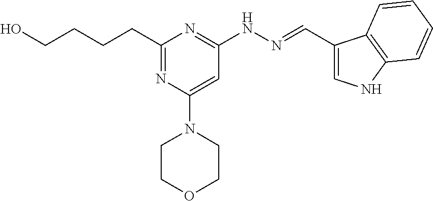 [SMILES: OCCCCc1nc(N/N=C/c2c[nH]c3ccccc23)cc(N2CCOCC2)n1] | E)-4-(4-(2-((1H-indol-3- yl)methylene)hydrazinyl)-6- morpholinopyrimidin-2-yl)butan-1-ol |
| 4 | 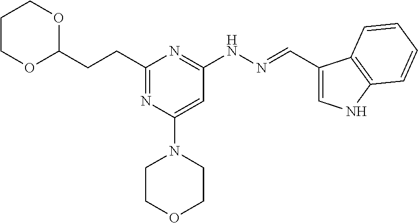 [SMILES: C(=N\Nc1cc(N2CCOCC2)nc(CCC2OCCCO2)n1)/c1c[nH]c2ccccc12] | (E)-4-(2-(2-(1,3-dioxan-2-yl)ethyl)-6-(2- ((1H-indol-3- yl)methylene)hydrazinyl)pyrimidin-4- yl)morpholine |
| 5 | 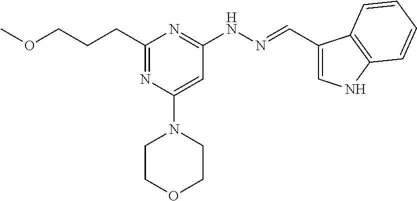 [SMILES: COCCCc1nc(N/N=C/c2c[nH]c3ccccc23)cc(N2CCOCC2)n1] | (E)-4-(6-(2-((1H-indol-3- yl)methylene)hydrazinyl)-2-(3- methoxypropyl)pyrimidin-4- yl)morpholine |
| 6 | 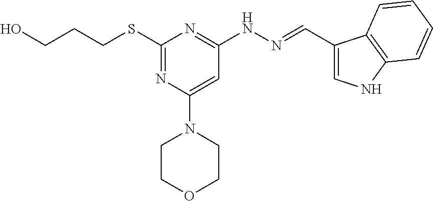 [SMILES: OCCCSc1nc(N/N=C/c2c[nH]c3ccccc23)cc(N2CCOCC2)n1] | (E)-3-((4-(2-((1H-indol-3- yl)methylene)hydrazinyl)-6- morpholinopyrimidin-2-yl)thio)propan-1- ol |
| 7 | 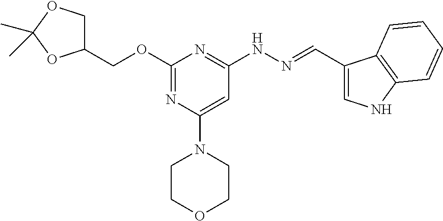 [SMILES: CC1(C)OCC(COc2nc(N/N=C/c3c[nH]c4ccccc34)cc(N3CCOCC3)n2)O1] | (E)-4-(6-(2-((1H-indol-3- yl)methylene)hydrazinyl)-2-((2,2- dimethyl-1,3-dioxolan-4- yl)methoxy)pyrimidin-4-yl)morpholine |
| 8 | 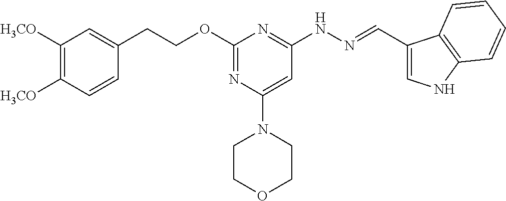 [SMILES: COc1ccc(CCOc2nc(N/N=C/c3c[nH]c4ccccc34)cc(N3CCOCC3)n2)cc1OC] | (E)-4-(6-(2-((1H-indol-3- yl)methylene)hydrazinyl)-2-(3,4- dimethoxyphenethoxy)pyrimidin-4- yl)morpholine |
| 9 | 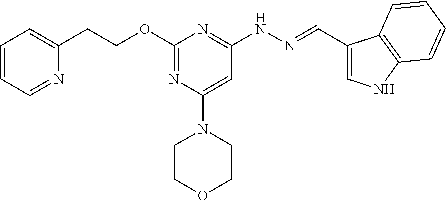 [SMILES: C(=N\Nc1cc(N2CCOCC2)nc(OCCc2ccccn2)n1)/c1c[nH]c2ccccc12] | (E)-4-(6-(2-((1H-indol-3- yl)methylene)hydrazinyl)-2-(2-(pyridin- 2-yl)ethoxy)pyrimidin-4-yl)morpholine |
| 10 | 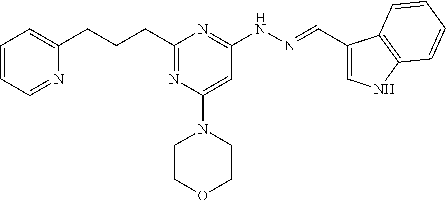 [SMILES: C(=N\Nc1cc(N2CCOCC2)nc(CCCc2ccccn2)n1)/c1c[nH]c2ccccc12] | (E)-4-(6-(2-((1H-indol-3- yl)methylene)hydrazinyl)-2-(3-(pyridin- 2-yl)propyl)pyrimidin-4-yl)morpholine |
| 11 | 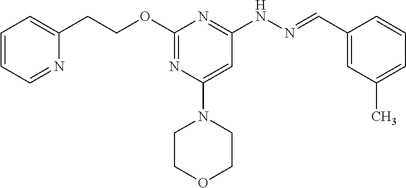 [SMILES: Cc1cccc(/C=N/Nc2cc(N3CCOCC3)nc(OCCc3ccccn3)n2)c1] | (E)-4-(6-(2-(3- methylbenzylidene)hydrazinyl)-2-(2- (pyridin-2-yl)ethoxy)pyrimidin-4- yl)morpholine |
| 12 | 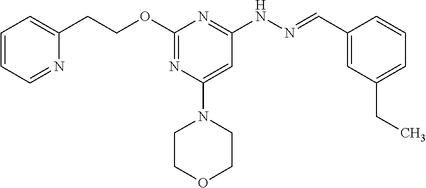 [SMILES: CCc1cccc(/C=N/Nc2cc(N3CCOCC3)nc(OCCc3ccccn3)n2)c1] | (E)-4-(6-(2-(3- ethylbenzylidene)hydrazinyl)-2-(2- (pyridin-2-yl)ethoxy)pyrimidin-4- yl)morpholine |
| 13 | 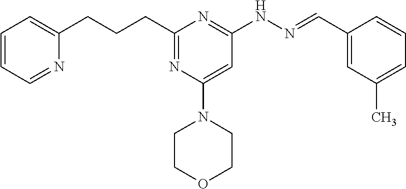 [SMILES: Cc1cccc(/C=N/Nc2cc(N3CCOCC3)nc(CCCc3ccccn3)n2)c1] | (E)-4-(6-(2-(3- methylbenzylidene)hydrazinyl)-2-(3- (pyridin-2-yl)propyl)pyrimidin-4- yl)morpholine |
| 14 | 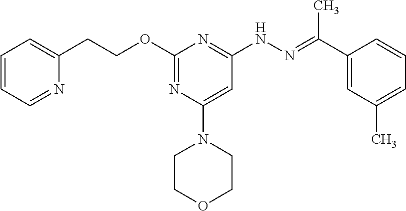 [SMILES: C/C(=N\Nc1cc(N2CCOCC2)nc(OCCc2ccccn2)n1)c1cccc(C)c1] | (E)-4-(2-(2-(pyridin-2-yl)ethoxy)-6-(2-(1- (m- tolyl)ethylidene)hydrazinyl)pyrimidin-4- yl)morpholine |
| 15 | 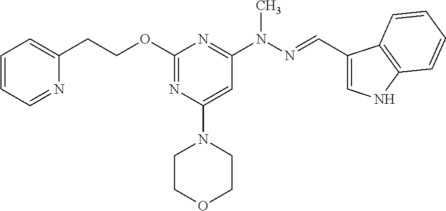 [SMILES: CN(/N=C/c1c[nH]c2ccccc12)c1cc(N2CCOCC2)nc(OCCc2ccccn2)n1] | (E)-4-(6-(2-((1H-indol-3-yl)methylene)- 1-methylhydrazinyl)-2-(2-(pyridin-2- yl)ethoxy)pyrimidin-4-yl)morpholine |
| 16 | 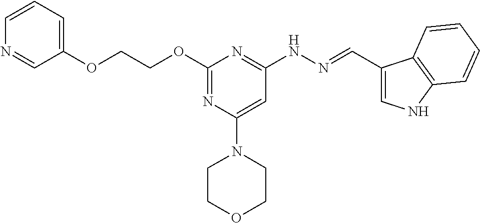 [SMILES: C(=N\Nc1cc(N2CCOCC2)nc(OCCOc2cccnc2)n1)/c1c[nH]c2ccccc12] | (E)-4-(6-(2-((1H-indol-3- yl)methylene)hydrazinyl)-2-(2-(pyridin- 3-yloxy)ethoxy)pyrimidin-4- yl)morpholine |
| 17 | 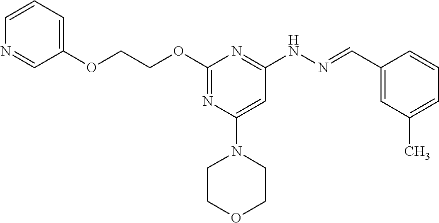 [SMILES: Cc1cccc(/C=N/Nc2cc(N3CCOCC3)nc(OCCOc3cccnc3)n2)c1] | (E)-4-(6-(2-(3- methylbenzylidene)hydrazinyl)-2-(2- (pyridin-3-yloxy)ethoxy)pyrimidin-4- yl)morpholine |
| 18 | 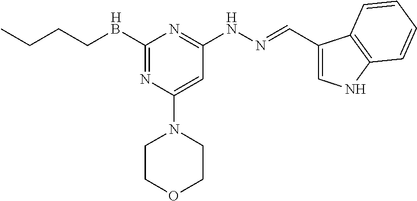 | (E)-4-(2-((1H-indol-3- yl)methylene)hydrazinyl)-N-butyl-6- morpholinopyrimidin-2-amine |
| 19 | 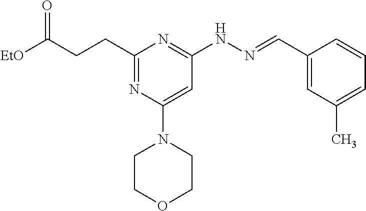 [SMILES: CCOC(=O)CCc1nc(N/N=C/c2cccc(C)c2)cc(N2CCOCC2)n1] | (E)-ethyl 3-(4-(2-(3- methylbenzylidene)hydrazinyl)-6- morpholinopyrimidin-2-yl)propanoate |
| 20 | 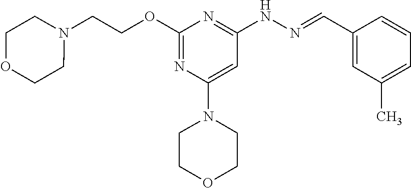 [SMILES: Cc1cccc(/C=N/Nc2cc(N3CCOCC3)nc(OCCN3CCOCC3)n2)c1] | (E)-4-(6-(2-(3- methylbenzylidene)hydrazinyl)-2-(2- morpholinoethoxy)pyrimidin-4- yl)morpholine |
| 21 | 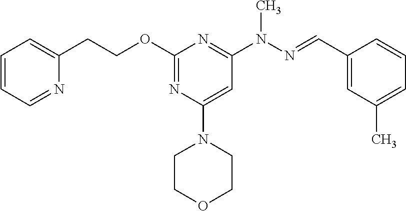 [SMILES: Cc1cccc(/C=N/N(C)c2cc(N3CCOCC3)nc(OCCc3ccccn3)n2)c1] | (E)-4-(6-(1-methyl-2-(3- methylbenzylidene)hydrazinyl)-2-(2- (pyridin-2-yl)ethoxy)pyrimidin-4- yl)morpholine |
| 22 | 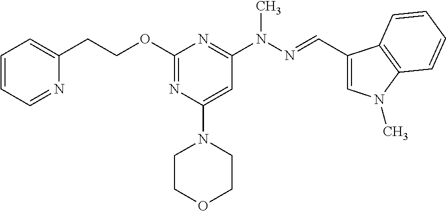 [SMILES: CN(/N=C/c1cn(C)c2ccccc12)c1cc(N2CCOCC2)nc(OCCc2ccccn2)n1] | (E)-4-(6-(1-methyl-2-((1-methyl-1H- indol-3-yl)methylene)hydrazinyl)-2-(2- (pyridin-2-yl)ethoxy)pyrimidin-4- yl)morpholine |
| 23 | 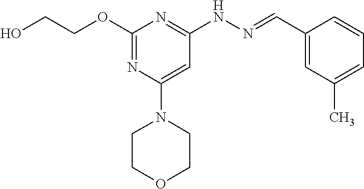 [SMILES: Cc1cccc(/C=N/Nc2cc(N3CCOCC3)nc(OCCO)n2)c1] | (E)-2-((4-(2-(3- methylbenzylidene)hydrazinyl)-6- morpholinopyrimidin-2-yl)oxy)ethanol |
| 24 | 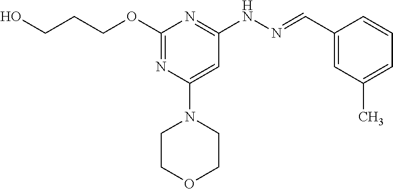 [SMILES: Cc1cccc(/C=N/Nc2cc(N3CCOCC3)nc(OCCCO)n2)c1] | (E)-3-((4-(2-(3- methylbenzylidene)hydrazinyl)-6- morpholinopyrimidin-2-yl)oxy)propan-1- ol |
| 25 | 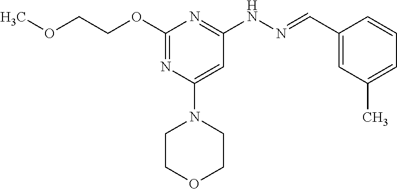 [SMILES: COCCOc1nc(N/N=C/c2cccc(C)c2)cc(N2CCOCC2)n1] | (E)-4-(2-(2-methoxyethoxy)-6-(2-(3- methylbenzylidene)hydrazinyl)pyrimidin- 4-yl)morpholine |
| 26 | 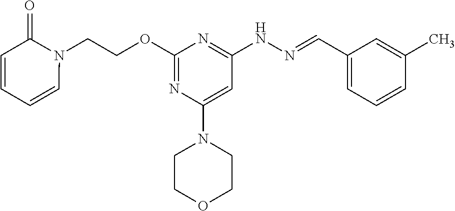 [SMILES: Cc1cccc(/C=N/Nc2cc(N3CCOCC3)nc(OCCn3ccccc3=O)n2)c1] | (E)-1-(2-((4-(2-(3- methylbenzylidene)hydrazinyl)-6- morpholinopyrimidin-2- yl)oxy)ethyl)pyridin-2(1H)-one |
| 27 | 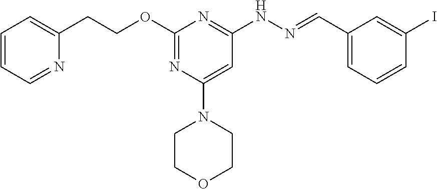 [SMILES: Ic1cccc(/C=N/Nc2cc(N3CCOCC3)nc(OCCc3ccccn3)n2)c1] | (E)-4-(6-(2-(3- iodobenzylidene)hydrazinyl)-2-(2- (pyridin-2-yl)ethoxy)pyrimidin-4- yl)morpholine |
| 28 | 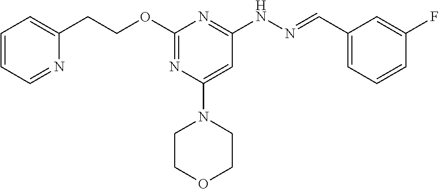 [SMILES: Fc1cccc(/C=N/Nc2cc(N3CCOCC3)nc(OCCc3ccccn3)n2)c1] | (E)-4-(6-(2-(3- fluorobenzylidene)hydrazinyl)-2-(2- (pyridin-2-yl)ethoxy)pyrimidin-4- yl)morpholine |
| 29 | 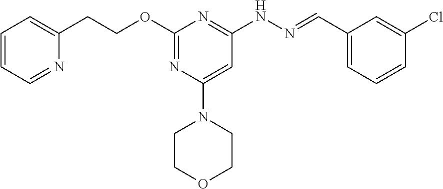 [SMILES: Clc1cccc(/C=N/Nc2cc(N3CCOCC3)nc(OCCc3ccccn3)n2)c1] | (E)-4-(6-(2-(3- fluorobenzylidene)hydrazinyl)-2-(2- (pyridin-2-yl)ethoxy)pyrimidin-4- yl)morpholine |
| 30 | 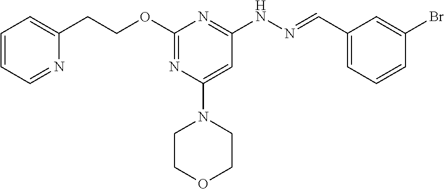 [SMILES: Brc1cccc(/C=N/Nc2cc(N3CCOCC3)nc(OCCc3ccccn3)n2)c1] | (E)-4-(6-(2-(3- bromobenzylidene)hydrazinyl)-2-(2- (pyridin-2-yl)ethoxy)pyrimidin-4- yl)morpholine |
| 31 | 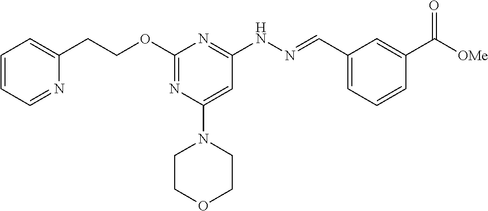 [SMILES: COC(=O)c1cccc(/C=N/Nc2cc(N3CCOCC3)nc(OCCc3ccccn3)n2)c1] | (E)-methyl 3-((2-(6-morpholino-2-(2- (pyridin-2-yl)ethoxy)pyrimidin-4- yl)hydrazono)methyl)benzoate |
| 32 | 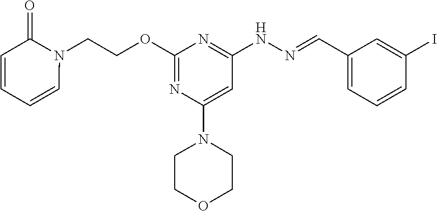 [SMILES: O=c1ccccn1CCOc1nc(N/N=C/c2cccc(I)c2)cc(N2CCOCC2)n1] | (E)-1-(2-((4-(2-(3- iodobenzylidene)hydrazinyl)-6- morpholinopyrimidin-2- yl)oxy)ethyl)pyridin-2(1H)-one |
| 33 | 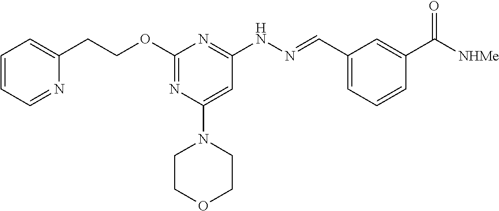 [SMILES: CNC(=O)c1cccc(/C=N/Nc2cc(N3CCOCC3)nc(OCCc3ccccn3)n2)c1] | (E)-N-methyl-3-((2-(6-morpholino-2-(2- (pyridin-2-yl)ethoxy)pyrimidin-4- yl)hydrazono)methyl)benzamide |
| 34 | 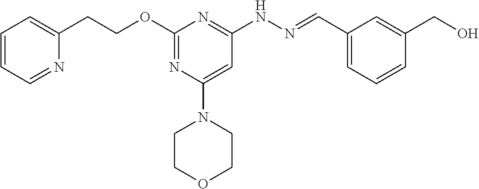 [SMILES: OCc1cccc(/C=N/Nc2cc(N3CCOCC3)nc(OCCc3ccccn3)n2)c1] | (E)-(3-((2-(6-morpholino-2-(2-(pyridin-2- yl)ethoxy)pyrimidin-4- yl)hydrazono)methyl)phenyl)methanol |

Further disclosed are methods for treating a lysosomal storage disorder in a subject in need thereof, comprising administering a compound, or pharmaceutically acceptable salt thereof, according to Formula (Ib):

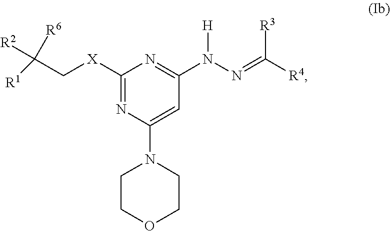

wherein
X is selected from the group consisting of: O and CH₂;
R¹ is selected from the group consisting of: C₁₋₃alkyl, oxazolidinonyl, morpholinyl, and pyridyl, wherein the C₁₋₃alkyl is optionally substituted with 1-2 substituents independently selected from the group consisting of: —OCH₃ and —OH;
R² is selected from the group consisting of: H and CH₃;
R³ and R⁴ taken together form a carbocyclic, aryl, or heteroaryl ring, each of which is optionally substituted with 1-2 substituents independently selected from the group consisting of: halo, oxo, —OH, C₁₋₄alkyl, —O(C₁₋₄alkyl), and —NO₂; and
R⁶ is selected from the group consisting of: H and CH₃.

In some embodiments, X is O.

In some embodiments, R¹ is morpholinyl.

In some embodiments, R² is H.

In some embodiments, R³ and R⁴ taken together form an aryl ring, optionally substituted with 1-2 substituents independently selected from the group consisting of: halo, oxo, —OH, C₁₋₄alkyl, —O(C₁₋₄alkyl), and —NO₂. In some embodiments, R³ and R⁴ taken together form a heteroaryl ring, optionally substituted with 1-2 substituents independently selected from the group consisting of: halo, oxo, —OH, and —O(C₁₋₄alkyl). In some embodiments, R³ and R⁴ taken together form a carbocyclic ring, optionally substituted with C₁₋₄alkyl.

In some embodiments, R⁶ is H.

In some embodiments, R² and R⁶ are H.

In some embodiments, the compound or pharmaceutically acceptable salt of Formula (Ib) is one selected from:

| Cmpd | | |
|---|---|---|
| # | Structure | Chemical Name |
| 35 | 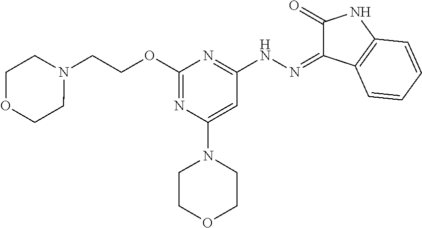 [SMILES: O=C1Nc2ccccc2/C1=N/Nc1cc(N2CCOCC2)nc(OCCN2CCOCC2)n1] | (Z)-3-(2-(6-morpholino-2-(2- morpholinoethoxy)pyrimidin-4- yl)hydrazono)indolin-2-one |
| 36 | 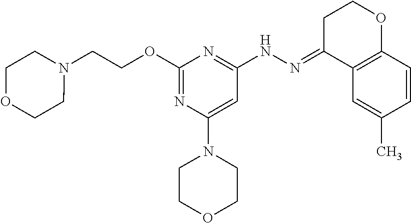 [SMILES: Cc1ccc2c(c1)/C(=N/Nc1cc(N3CCOCC3)nc(OCCN3CCOCC3)n1)CCO2] | (E)-4-(6-(2-(6-methylchroman-4- ylidene)hydrazinyl)-2-(2- morpholinoethoxy)pyrimidin-4- yl)morpholine |
| 37 | 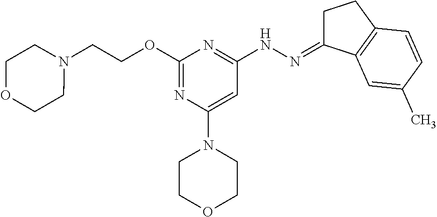 [SMILES: Cc1ccc2c(c1)/C(=N/Nc1cc(N3CCOCC3)nc(OCCN3CCOCC3)n1)CC2] | (E)-4-(6-(2-(6-methyl-2,3-dihydro- 1H-inden-1-ylidene)hydrazinyl)-2- (2-morpholinoethoxy)pyrimidin-4- yl)morpholine |
| 38 | 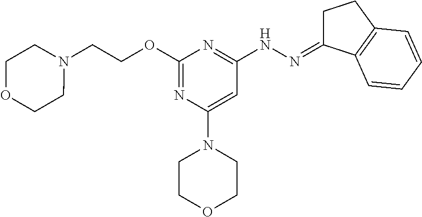 [SMILES: c1ccc2c(c1)CC/C2=N\Nc1cc(N2CCOCC2)nc(OCCN2CCOCC2)n1] | (E)-4-(6-(2-(2,3-dihydro-1H-inden- 1-ylidene)hydrazinyl)-2-(2- morpholinoethoxy)pyrimidin-4- yl)morpholine |
| 39 | 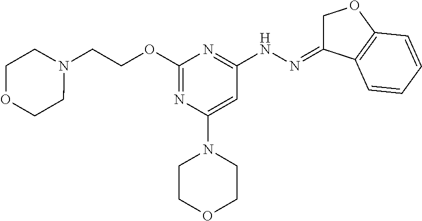 [SMILES: c1ccc2c(c1)OC/C2=N\Nc1cc(N2CCOCC2)nc(OCCN2CCOCC2)n1] | (Z)-4-(6-(2-(benzofuran-3(2H)- ylidene)hydrazinyl)-2-(2- morpholinoethoxy)pyrimidin-4- yl)morpholine |
| 40 | 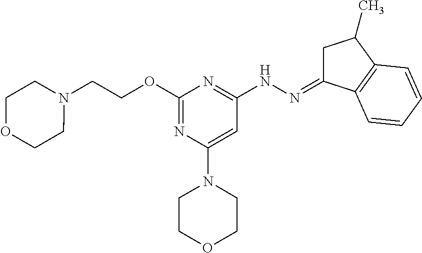 [SMILES: CC1C/C(=N\Nc2cc(N3CCOCC3)nc(OCCN3CCOCC3)n2)c2ccccc21] | (E)-4-(6-(2-(3-methyl-2,3-dihydro- 1H-inden-1-ylidene)hydrazinyl)-2- (2-morpholinoethoxy)pyrimidin-4- yl)morpholine |
| 41 | 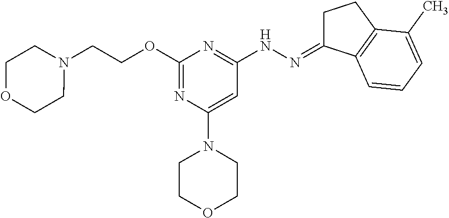 [SMILES: Cc1cccc2c1CC/C2=N\Nc1cc(N2CCOCC2)nc(OCCN2CCOCC2)n1] | (E)-4-(6-(2-(4-methyl-2,3-dihydro- 1H-inden-1-ylidene)hydrazinyl)-2- (2-morpholinoethoxy)pyrimidin-4- yl)morpholine |
| 42 | 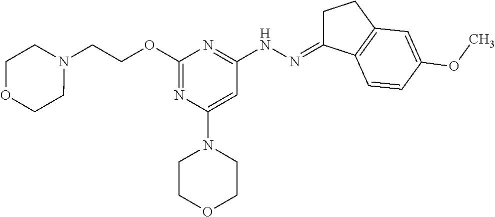 [SMILES: COc1ccc2c(c1)CC/C2=N\Nc1cc(N2CCOCC2)nc(OCCN2CCOCC2)n1] | (E)-4-(6-(2-(5-methoxy-2,3- dihydro-1H-inden-1- ylidene)hydrazinyl)-2-(2- morpholinoethoxy)pyrimidin-4- yl)morpholine |
| 43 | 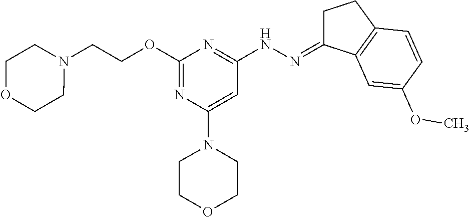 [SMILES: COc1ccc2c(c1)/C(=N/Nc1cc(N3CCOCC3)nc(OCCN3CCOCC3)n1)CC2] | (E)-4-(6-(2-(6-methoxy-2,3- dihydro-1H-inden-1- ylidene)hydrazinyl)-2-(2- morpholinoethoxy)pyrimidin-4- yl)morpholine |
| 44 | 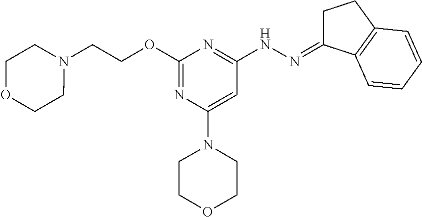 [SMILES: c1ccc2c(c1)CC/C2=N\Nc1cc(N2CCOCC2)nc(OCCN2CCOCC2)n1] | (E)-4-(6-(2-(2,3-dihydro-1H-inden- 1-ylidene)hydrazinyl)-2-(2- morpholinoethoxy)pyrimidin-4- yl)morpholine |
| 45 | 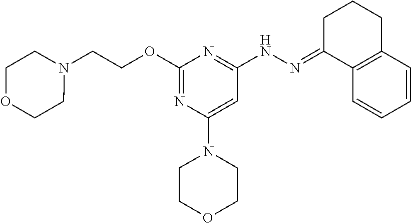 [SMILES: c1ccc2c(c1)CCC/C2=N\Nc1cc(N2CCOCC2)nc(OCCN2CCOCC2)n1] | (E)-4-(6-(2-(3,4-dihydronaphthalen- 1(2H)-ylidene)hydrazinyl)-2-(2- morpholinoethoxy)pyrimidin-4- yl)morpholine |
| 46 | 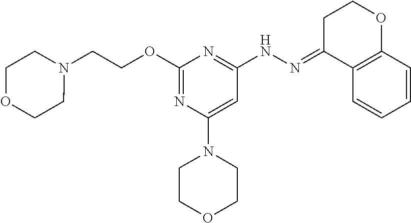 [SMILES: c1ccc2c(c1)OCC/C2=N\Nc1cc(N2CCOCC2)nc(OCCN2CCOCC2)n1] | (E)-4-(6-(2-(chroman-4- ylidene)hydrazinyl)-2-(2- morpholinoethoxy)pyrimidin-4- yl)morpholine |
| 47 | 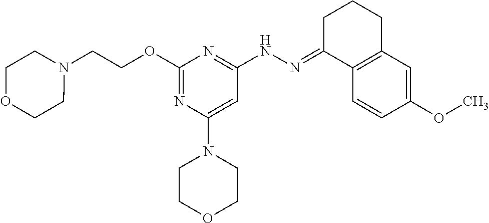 [SMILES: COc1ccc2c(c1)CCC/C2=N\Nc1cc(N2CCOCC2)nc(OCCN2CCOCC2)n1] | (E)-4-(6-(2-(6-methoxy-3,4- dihydronaphthalen-1(2H)- ylidene)hydrazinyl)-2-(2- morpholinoethoxy)pyrimidin-4- yl)morpholine |
| 48 | 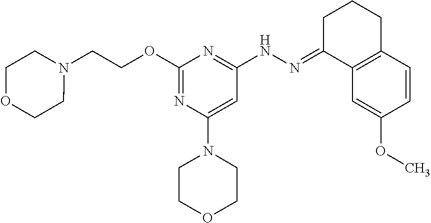 [SMILES: COc1ccc2c(c1)/C(=N/Nc1cc(N3CCOCC3)nc(OCCN3CCOCC3)n1)CCC2] | (E)-4-(6-(2-(7-methoxy-3,4- dihydronaphthalen-1(2H)- ylidene)hydrazinyl)-2-(2- morpholinoethoxy)pyrimidin-4- yl)morpholine |
| 49 | 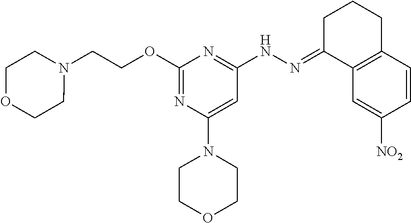 [SMILES: O=[N+]([O-])c1ccc2c(c1)/C(=N/Nc1cc(N3CCOCC3)nc(OCCN3CCOCC3)n1)CCC2] | (E)-4-(2-((4-morpholino-6-(2-(7- nitro-3,4-dihydronaphthalen-1(2H)- ylidene)hydrazinyl)pyrimidin-2- yl)oxy)ethyl)morpholine |
| 50 | 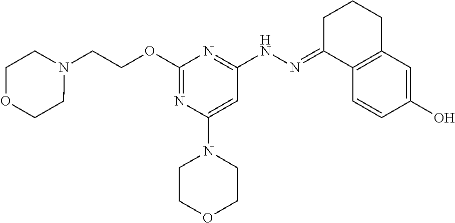 [SMILES: Oc1ccc2c(c1)CCC/C2=N\Nc1cc(N2CCOCC2)nc(OCCN2CCOCC2)n1] | (E)-5-(2-(6-morpholino-2-(2- morpholinoethoxy)pyrimidin-4- yl)hydrazono)-5,6,7,8- tetrahydronaphthalen-2-ol |
| 51 | 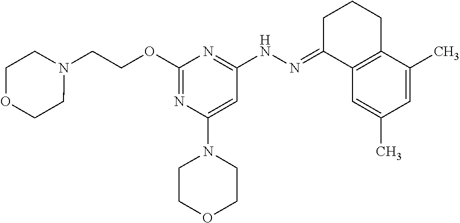 [SMILES: Cc1cc(C)c2c(c1)/C(=N/Nc1cc(N3CCOCC3)nc(OCCN3CCOCC3)n1)CCC2] | (E)-4-(6-(2-(5,7-dimethyl-3,4- dihydronaphthalen-1(2H)- ylidene)hydrazinyl)-2-(2- morpholinoethoxy)pyrimidin-4- yl)morpholine |
| 52 | 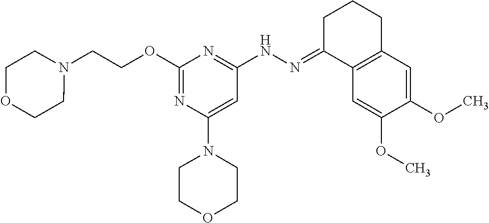 [SMILES: COc1cc2c(cc1OC)/C(=N/Nc1cc(N3CCOCC3)nc(OCCN3CCOCC3)n1)CCC2] | (E)-4-(6-(2-(6,7-dimethoxy-3,4- dihydronaphthalen-1(2H)- ylidene)hydrazinyl)-2-(2- morpholinoethoxy)pyrimidin-4- yl)morpholine |
| 53 | 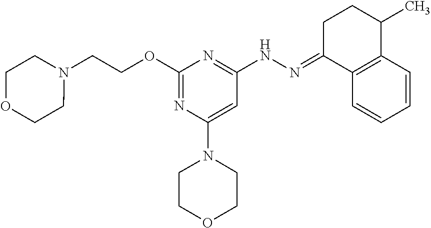 [SMILES: CC1CC/C(=N\Nc2cc(N3CCOCC3)nc(OCCN3CCOCC3)n2)c2ccccc21] | (E)-4-(6-(2-(4-methyl-3,4- dihydronaphthalen-1(2H)- ylidene)hydrazinyl)-2-(2- morpholinoethoxy)pyrimidin-4- yl)morpholine |
| 54 | 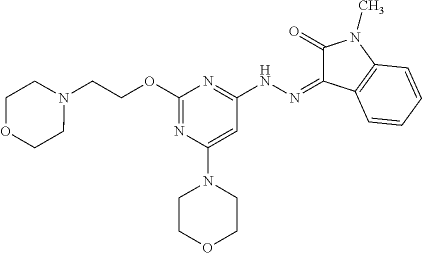 [SMILES: CN1C(=O)/C(=N\Nc2cc(N3CCOCC3)nc(OCCN3CCOCC3)n2)c2ccccc21] | (Z)-1-methyl-3-(2-(6-morpholino-2- (2-morpholinoethoxy)pyrimidin-4- yl)hydrazono)indolin-2-one |
| 55 | 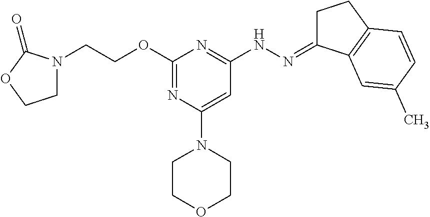 [SMILES: Cc1ccc2c(c1)/C(=N/Nc1cc(N3CCOCC3)nc(OCCN3CCOC3=O)n1)CC2] | (E)-3-(2-((4-(2-(6-methyl-2,3- dihydro-1H-inden-1- ylidene)hydrazinyl)-6- morpholinopyrimidin-2- yl)oxy)ethyl)oxazolidin-2-one |
| 56 | 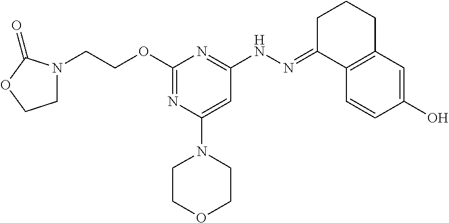 [SMILES: O=C1OCCN1CCOc1nc(N/N=C2\CCCc3cc(O)ccc32)cc(N2CCOCC2)n1] | (E)-3-(2-((4-(2-(6-hydroxy-3,4- dihydronaphthalen-1(2H)- ylidene)hydrazinyl)-6- morpholinopyrimidin-2- yl)oxy)ethyl)oxazolidin-2-one |
| 57 | 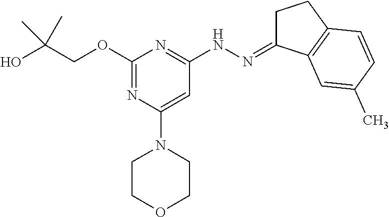 [SMILES: Cc1ccc2c(c1)/C(=N/Nc1cc(N3CCOCC3)nc(OCC(C)(C)O)n1)CC2] | (E)-2-methyl-4-(4-(2-(6-methyl-2,3- dihydro-1H-inden-1- ylidene)hydrazinyl)-6- morpholinopyrimidin-2-yl)butan-2- ol |
| 58 | 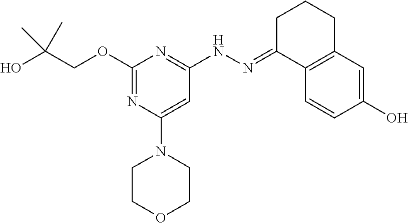 [SMILES: CC(C)(O)COc1nc(N/N=C2\CCCc3cc(O)ccc32)cc(N2CCOCC2)n1] | (E)-5-(2-(2-(3-hydroxy-3- methylbutyl)-6- morpholinopyrimidin-4- yl)hydrazono)-5,6,7,8- tetrahydronaphthalen-2-ol |
| 59 | 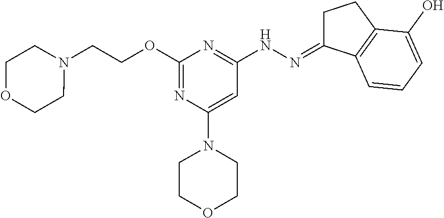 [SMILES: Oc1cccc2c1CC/C2=N\Nc1cc(N2CCOCC2)nc(OCCN2CCOCC2)n1] | (E)-1-(2-(6-morpholino-2-(2- morpholinoethoxy)pyrimidin-4- yl)hydrazono)-2,3-dihydro-1H- inden-4-ol |
| 60 | 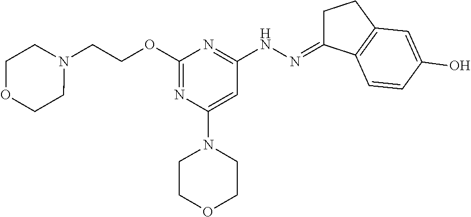 [SMILES: Oc1ccc2c(c1)CC/C2=N\Nc1cc(N2CCOCC2)nc(OCCN2CCOCC2)n1] | (E)-1-(2-(6-morpholino-2-(2- morpholinoethoxy)pyrimidin-4- yl)hydrazono)-2,3-dihydro-1H- inden-5-ol |
| 61 | 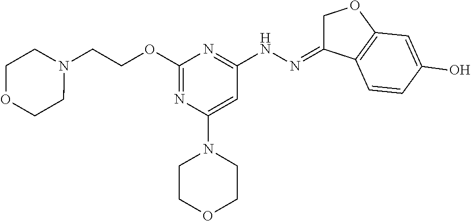 [SMILES: Oc1ccc2c(c1)OC/C2=N\Nc1cc(N2CCOCC2)nc(OCCN2CCOCC2)n1] | (Z)-3-(2-(6-morpholino-2-(2- morpholinoethoxy)pyrimidin-4- yl)hydrazono)-2,3- dihydrobenzofuran-6-ol |
| 62 | 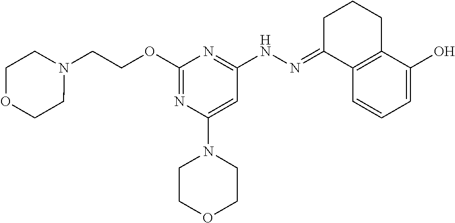 [SMILES: Oc1cccc2c1CCC/C2=N\Nc1cc(N2CCOCC2)nc(OCCN2CCOCC2)n1] | (E)-5-(2-(6-morpholino-2-(2- morpholinoethoxy)pyrimidin-4- yl)hydrazono)-5,6,7,8- tetrahydronaphthalen-1-ol |
| 63 | 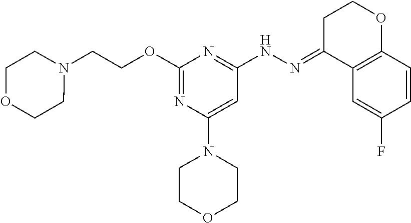 [SMILES: Fc1ccc2c(c1)/C(=N/Nc1cc(N3CCOCC3)nc(OCCN3CCOCC3)n1)CCO2] | (E)-4-(6-(2-(6-fluorochroman-4- ylidene)hydrazinyl)-2-(2- morpholinoethoxy)pyrimidin-4- yl)morpholine |
| 64 | 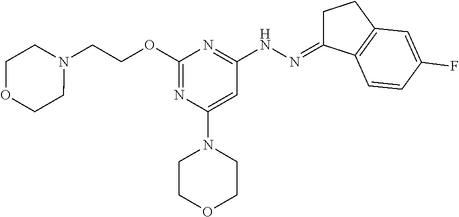 [SMILES: Fc1ccc2c(c1)CC/C2=N\Nc1cc(N2CCOCC2)nc(OCCN2CCOCC2)n1] | (E)-4-(6-(2-(5-fluoro-2,3-dihydro- 1H-inden-1-ylidene)hydrazinyl)-2- (2-morpholinoethoxy)pyrimidin-4- yl)morpholine |
| 65 | 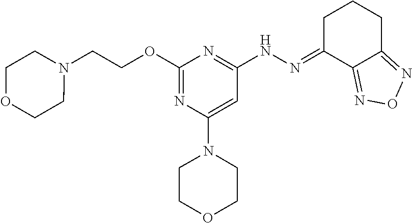 [SMILES: c1c(N/N=C2\CCCc3nonc32)nc(OCCN2CCOCC2)nc1N1CCOCC1] | (E)-4-(2-(6-morpholino-2-(2- morpholinoethoxy)pyrimidin-4- yl)hydrazono)-4,5,6,7- tetrahydrobenzo[c][1,2,5]oxadiazole |
| 66 | 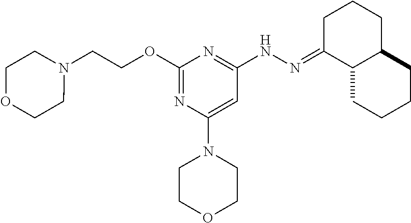 | 4-(2-((4-morpholino-6-((E)-2- ((4aR,8aS)-octahydronaphthalen- 1(2H)- ylidene)hydrazinyl)pyrimidin-2- yl)oxy)ethyl)morpholine |
| 67 | 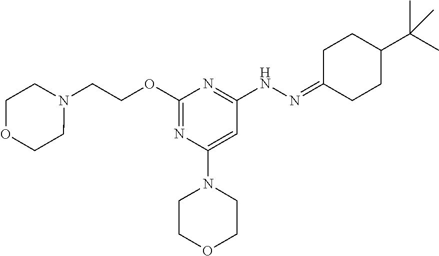 [SMILES: CC(C)(C)C1CCC(=NNc2cc(N3CCOCC3)nc(OCCN3CCOCC3)n2)CC1] | 4-(6-(2-(4-(tert- butyl)cyclohexylidene)hydrazinyl)- 2-(2-morpholinoethoxy)pyrimidin- 4-yl)morpholine |
| 68 | 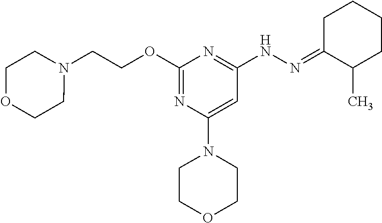 [SMILES: CC1CCCC/C1=N\Nc1cc(N2CCOCC2)nc(OCCN2CCOCC2)n1] | (E)-4-(6-(2-(2- methylcyclohexylidene)hydrazinyl)- 2-(2-morpholinoethoxy)pyrimidin- 4-yl)morpholine |
| 69 | 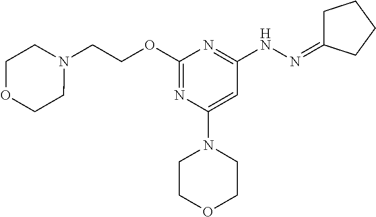 [SMILES: c1c(NN=C2CCCC2)nc(OCCN2CCOCC2)nc1N1CCOCC1] | 4-(6-(2- cyclopentylidenehydrazinyl)-2-(2- morpholinoethoxy)pyrimidin-4- yl)morpholine |
| 70 | 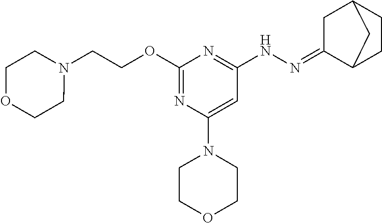 [SMILES: c1c(N/N=C2\CC3CCC2C3)nc(OCCN2CCOCC2)nc1N1CCOCC1] | (E)-4-(6-(2-(bicyclo[2.2.1]heptan-2- ylidene)hydrazinyl)-2-(2- morpholinoethoxy)pyrimidin-4- yl)morpholine |
| 71 | 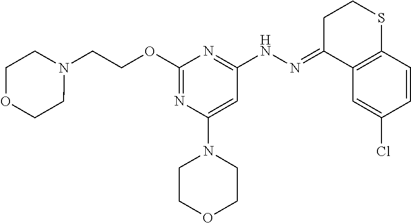 [SMILES: Clc1ccc2c(c1)/C(=N/Nc1cc(N3CCOCC3)nc(OCCN3CCOCC3)n1)CCS2] | (E)-4-(6-(2-(6-chlorothiochroman- 4-ylidene)hydrazinyl)-2-(2- morpholinoethoxy)pyrimidin-4- yl)morpholine |
| 72 | 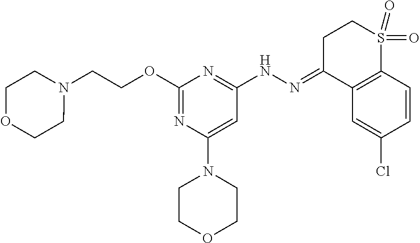 [SMILES: O=S1(=O)CC/C(=N\Nc2cc(N3CCOCC3)nc(OCCN3CCOCC3)n2)c2cc(Cl)ccc21] | (E)-6-chloro-4-(2-(6-morpholino-2- (2-morpholinoethoxy)pyrimidin-4- yl)hydrazono)thiochroman 1,1- dioxide |
| 73 | 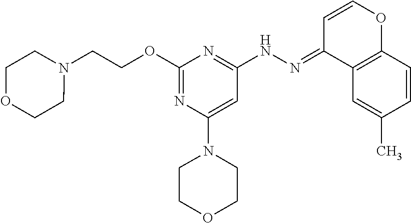 [SMILES: Cc1ccc2occ/c(=N\Nc3cc(N4CCOCC4)nc(OCCN4CCOCC4)n3)c2c1] | (E)-4-(6-(2-(6-methyl-4H-chromen- 4-ylidene)hydrazinyl)-2-(2- morpholinoethoxy)pyrimidin-4- yl)morpholine |
| 74 | 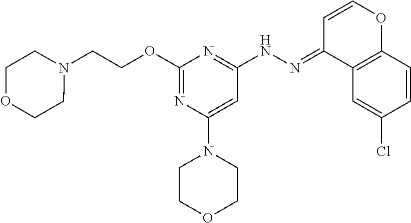 [SMILES: Clc1ccc2occ/c(=N\Nc3cc(N4CCOCC4)nc(OCCN4CCOCC4)n3)c2c1] | (E)-4-(6-(2-(6-chloro-4H-chromen- 4-ylidene)hydrazinyl)-2-(2- morpholinoethoxy)pyrimidin-4- yl)morpholine |
| 75 | 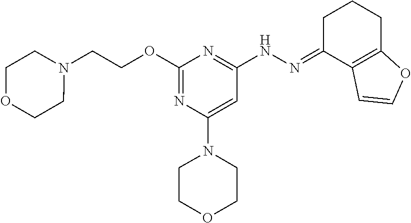 [SMILES: c1cc2c(o1)CCC/C2=N\Nc1cc(N2CCOCC2)nc(OCCN2CCOCC2)n1] | (E)-4-(6-(2-(6,7- dihydrobenzofuran-4(5H)- ylidene)hydrazinyl)-2-(2- morpholinoethoxy)pyrimidin-4- yl)morpholine |

In some embodiments, the compound of Formula (I) and/or (Ia) is 3-methylbenzaldehyde 2-[6-(4-Morpholinyl)-2-[2-(2-pyridinyl)ethoxy]-4-pyrimidinyl]hydrazone, or a pharmaceutically acceptable salt thereof, having the structure:

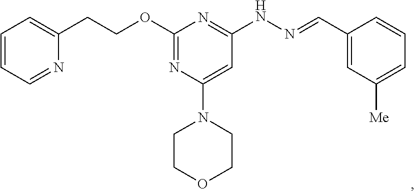 [SMILES: Cc1cccc(/C=N/Nc2cc(N3CCOCC3)nc(OCCc3ccccn3)n2)c1 ; ","]

The following paragraphs present a number of embodiments of methods for treating a lysosomal storage disorder in a subject in need thereof, comprising administering the compounds disclosed herein. In each instance the embodiment includes the recited compound(s) as well as a single stereoisomer or mixture of stereoisomers thereof, as well as a pharmaceutically acceptable salt thereof. The compounds include the N-oxides or pharmaceutically acceptable salts thereof. In some situations, the compounds exist as tautomers. All tautomers are included within the scope of the compounds presented herein.

In some or any embodiments, provided are methods for treating a lysosomal storage disorder in a subject in need thereof, comprising administering a pharmaceutical composition, wherein the pharmaceutical composition comprises 1) a Compound of Formula (I), (Ia), and/or (Ib) optionally as a tautomer, a single stereoisomer or mixture of stereoisomers thereof and additionally optionally as a pharmaceutically acceptable salt thereof, and 2) a pharmaceutically acceptable excipient or pharmaceutically acceptable carrier.

In some or any embodiments, optionally in combination with any or all of the above various embodiments, provided herein is a method for treating a lysosomal storage disorder in a subject in need thereof, comprising administering a pharmaceutical composition, wherein the pharmaceutical composition comprises a compound of Formula (I), (Ia), and/or (Ib), or stereoisomers, or a pharmaceutically acceptable salt, and one or more pharmaceutically acceptable carrier(s), excipient(s), binder(s) or diluent(s). In some or any embodiments, provided herein is a method using a pharmaceutical composition comprising of a compound of Formula (I), (Ia), and/or (Ib) or stereoisomers, or a pharmaceutically acceptable salt, and one or more pharmaceutically acceptable excipient(s). The pharmaceutical compositions can, moreover, contain preservatives, solubilizers, stabilizers, wetting agents, emulsifiers, sweeteners, colorants, flavorants, salts for varying the osmotic pressure, buffers, coating agents or antioxidants.

In some or any embodiments, disclosed is a method described herein using a pharmaceutical composition comprising a compound, pharmaceutically acceptable salt, pharmaceutically acceptable N-oxide, pharmaceutically active metabolite, pharmaceutically acceptable prodrug, or pharmaceutically acceptable solvate of any of the compounds disclosed herein. In some embodiments, the pharmaceutical compositions further comprises a pharmaceutically acceptable diluent, excipient or binder.

In some embodiments, the pharmaceutically acceptable salt is selected from the group consisting of: hydrochloric acid, nitric acid, sulfuric acid, hydrobromic acid, hydroiodic acid, perchloric acid, phosphoric acid, alkylsulfonic acids, aryl sulfonic acids, halogenated alkylsulfonic acids, halogenated acetic acids, picric acid, oxalic acid, citric acid, formic acid, ascorbic acid and benzoic acid. For example, the pharmaceutically acceptable salt can be a methanesulfonic acid salt. In some embodiments, the pharmaceutically acceptable salt is a hydrobromic acid salt. In some embodiments, the pharmaceutically acceptable salt is a hydrochloric acid salt.

In some or any embodiments, pharmaceutical compositions are formulated in any manner, including using one or more physiologically acceptable carriers comprising excipients and/or auxiliaries which facilitate processing of the active compounds into pharmaceutical preparations. In some embodiments, proper formulation is dependent upon the route of administration chosen. In various embodiments, any techniques, carriers, and excipients are used as suitable.

A pharmaceutical composition, as used herein, refers to a mixture of a compound described herein with other chemical components, such as carriers, stabilizers, diluents, dispersing agents, suspending agents, thickening agents, and/or excipients. In some or any embodiments, a pharmaceutical composition facilitates administration of the compound to an organism. In some embodiments, practicing the methods of treatment or use provided herein, includes administering or using a pharmaceutical composition comprising a therapeutically effective amount of a compound provided herein. In specific embodiments, the methods of treatment provided for herein include administering such a pharmaceutical composition to a mammal having a disease or condition to be treated. In one embodiment, the mammal is a human. In some embodiments, the therapeutically effective amount varies widely depending on the severity of the disease, the age and relative health of the subject, the potency of the compound used and other factors. In various embodiments, the compounds described herein are used singly or in combination with one or more therapeutic agents as components of mixtures.

In some or any embodiments, it is provided a method of forming a composition, comprising providing a compound and forming the composition. In some embodiments, the composition further comprises a pharmaceutically acceptable carrier. In some embodiments of the composition, optionally in combination with any or all of the above various embodiments, the composition is formulated in a formulation for local or systemic delivery. Examples of such formulations are formulations for oral administration, injection, topical administration, pulmonary administration, or implant. In some or any embodiments, the compound is according to any of the various embodiments described above or below.

In some or any embodiments, the pharmaceutical compositions provided herein are formulated for intravenous injections. In certain aspects, the intravenous injection formulations provided herein are formulated as aqueous solutions, and, in some embodiments, in physiologically compatible buffers such as Hank's solution, Ringer's solution, or physiological saline buffer. In certain embodiments, the pharmaceutical compositions provided herein are formulated for transmucosal administration. In some aspects, transmucosal formulations include penetrants appropriate to the barrier to be permeated. In certain embodiments, the pharmaceutical compositions provided herein are formulated for other parenteral injections, appropriate formulations include aqueous or nonaqueous solutions, and in one embodiment, with physiologically compatible buffers or excipients.

In some or any embodiments, the pharmaceutical compositions provided herein are formulated for oral administration. In certain aspects, the oral formulations provided herein comprise compounds described herein that are formulated with pharmaceutically acceptable carriers or excipients. Such carriers enable the compounds described herein to be formulated as tablets, powders, pills, dragees, capsules, liquids, gels, syrups, elixirs, slurries, suspensions and the like, for oral ingestion by a patient to be treated.

In some or any embodiments, pharmaceutical preparations for oral use are obtained by mixing one or more solid excipient with one or more of the compounds described herein, optionally grinding the resulting mixture, and processing the mixture of granules, after adding suitable auxiliaries, if desired, to obtain tablets or dragee cores. Suitable excipients include, in particular, fillers such as sugars, including lactose, sucrose, mannitol, or sorbitol; cellulose preparations such as: for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents are optionally added, such as the cross-linked croscarmellose sodium, polyvinylpyrrolidone, agar, or alginic acid or a salt thereof such as sodium alginate.

In some or any embodiments, provided herein is a pharmaceutical composition formulated as dragee cores with suitable coatings. In some or any embodiments, concentrated sugar solutions are used in forming the suitable coating, and optionally contain gum arabic, talc, polyvinylpyrrolidone, carbopol gel, polyethylene glycol, and/or titanium dioxide, lacquer solutions, and suitable organic solvents or solvent mixtures. In some embodiments, dyestuffs and/or pigments are added to tablets, dragees and/or the coatings thereof for, e.g., identification or to characterize different combinations of active compound doses.

In some or any embodiments, pharmaceutical preparations which are used include orally include push-fit capsules made of gelatin, as well as soft, sealed capsules made of gelatin and a plasticizer, such as glycerol or sorbitol. In some embodiments, the push-fit capsules contain the active ingredients in admixture with filler such as lactose, binders such as starches, and/or lubricants such as talc or magnesium stearate and, optionally, stabilizers. In some or any embodiments, in soft capsules, the active compounds are dissolved or suspended in suitable liquids, such as fatty oils, liquid paraffin, or liquid polyethylene glycols. In addition, stabilizers are optionally added. In some or any embodiments, the formulations for oral administration are in dosages suitable for such administration.

In some or any embodiments, the pharmaceutical compositions provided herein are formulated for buccal or sublingual administration. In some or any embodiments, buccal or sublingual compositions take the form of tablets, lozenges, or gels formulated in a conventional manner. In some or any embodiments, parenteral injections involve bolus injection or continuous infusion. In some embodiments, formulations for injection are presented in unit dosage form, e.g., in ampoules or in multi-dose containers, with an added preservative. In some embodiments, the pharmaceutical composition described herein is in a form suitable for parenteral injection as a sterile suspensions, solutions or emulsions in oily or aqueous vehicles, and optionally contains formulatory agents such as suspending, stabilizing and/or dispersing agents. Pharmaceutical formulations for parenteral administration include aqueous solutions of the active compounds in water-soluble form. In some embodiments, suspensions of the active compounds are prepared as appropriate oily injection suspensions. Suitable lipophilic solvents or vehicles include fatty oils such as sesame oil, or synthetic fatty acid esters, such as ethyl oleate or triglycerides, or liposomes. In some or any embodiments, aqueous injection suspensions contain substances which increase the viscosity of the suspension, such as sodium carboxymethyl cellulose, sorbitol, or dextran. Optionally, the suspensions also contain suitable stabilizers or agents which increase the solubility of the compounds to allow for the preparation of highly concentrated solutions. In alternative embodiments, the active ingredient is in powder form for constitution with a suitable vehicle, e.g., sterile pyrogen-free water, before use.

In some embodiments, the compounds described herein are administered topically. In specific embodiments, the compounds described herein are formulated into a variety of topically administrable compositions, such as solutions, suspensions, lotions, gels, pastes, medicated sticks, balms, creams or ointments. Such pharmaceutical compounds optionally contain solubilizers, stabilizers, tonicity enhancing agents, buffers and/or preservatives.

In some or any embodiments, the pharmaceutical compositions provided herein are formulated for transdermal administration of compounds described herein. In some embodiments, administration of such compositions employs transdermal delivery devices and transdermal delivery patches. In some or any embodiments, the compositions are lipophilic emulsions or buffered, aqueous solutions, dissolved and/or dispersed in a polymer or an adhesive. Such patches include those constructed for continuous, pulsatile, or on demand delivery of pharmaceutical agents. In some embodiments, transdermal delivery of the compounds described herein is accomplished by use of iontophoretic patches and the like. In some or any embodiments, transdermal patches provide controlled delivery of the compounds provided herein, such as, for example, a compound of Formula (I). In some or any embodiments, the rate of absorption is slowed by using rate-controlling membranes or by trapping the compound within a polymer matrix or gel. Conversely, absorption enhancers are optionally used to increase absorption. Absorption enhancer and carrier include absorbable pharmaceutically acceptable solvents that assist in passage of the compound through the skin. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin.

In some or any embodiments, the pharmaceutical compositions provided herein are formulated for administration by inhalation. In some or any embodiments, in such pharmaceutical compositions formulated for inhalation, the compounds described herein are in a form as an aerosol, a mist or a powder. In some embodiments, pharmaceutical compositions described herein are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In certain aspects of a pressurized aerosol, the dosage unit is determined by providing a valve to deliver a metered amount. In some or any embodiments, capsules and cartridges of, such as, by way of example only, gelatin for use in an inhaler or insufflator is formulated containing a powder mix of the compound described herein and a suitable powder base such as lactose or starch.

In some embodiments, the compounds described herein are formulated in rectal compositions such as enemas, rectal gels, rectal foams, rectal aerosols, suppositories, jelly suppositories, or retention enemas. In some or any embodiments, rectal compositions optionally contain conventional suppository bases such as cocoa butter or other glycerides, as well as synthetic polymers such as polyvinylpyrrolidone, PEG, and the like. In some or any suppository forms of the compositions, a low-melting wax such as, but not limited to, a mixture of fatty acid glycerides, optionally in combination with cocoa butter is first melted.

In various embodiments provided herein, the pharmaceutical compositions are formulated in a conventional manner using one or more physiologically acceptable carriers comprising excipients and auxiliaries which facilitate processing of the active compounds into pharmaceutically acceptable preparations. In some or any embodiments, proper formulation is dependent upon the route of administration chosen. In various embodiments, any of the techniques, carriers, and excipients is used as suitable. In some embodiments, pharmaceutical compositions comprising a compound described herein are manufactured in a conventional manner, such as, by way of example only, by means of conventional mixing, dissolving, granulating, dragee-making, levigating, emulsifying, encapsulating, entrapping or compression processes.

In some or any embodiments, the pharmaceutical compositions include at least one pharmaceutically acceptable carrier, diluent or excipient and a compound described herein described herein as an active ingredient in free-acid or free-base form, or in a pharmaceutically acceptable salt form. In addition, the methods and pharmaceutical compositions described herein include the use of N-oxides, crystalline forms (also known as polymorphs), as well as active metabolites of these compounds having the same type of activity. In some situations, compounds described herein exist as tautomers. All tautomers are included within the scope of the compounds presented herein. Additionally, included herein are the solvated and unsolvated forms of the compounds described herein. Solvated compounds include those that are solvated with pharmaceutically acceptable solvents such as water, ethanol, and the like. The solvated forms of the compounds presented herein are also considered to be disclosed herein. In some embodiments, the pharmaceutical compositions described herein include other medicinal or pharmaceutical agents, carriers, adjuvants, such as preserving, stabilizing, wetting or emulsifying agents, solution promoters, salts for regulating the osmotic pressure, and/or buffers. In additional embodiments, the pharmaceutical compositions described herein also contain other therapeutically valuable substances.

Methods for the preparation of compositions containing the compounds described herein include formulating the compounds with one or more inert, pharmaceutically acceptable excipients or carriers to form a solid, semi-solid or liquid. Solid compositions include, but are not limited to, powders, tablets, dispersible granules, capsules, cachets, and suppositories. Liquid compositions include solutions in which a compound is dissolved, emulsions comprising a compound, or a solution containing liposomes, micelles, or nanoparticles comprising a compound as disclosed herein. Semi-solid compositions include, but are not limited to, gels, suspensions and creams. In various embodiments, the compositions are in liquid solutions or suspensions, solid forms suitable for solution or suspension in a liquid prior to use, or as emulsions. These compositions optionally contain minor amounts of nontoxic, auxiliary substances, such as wetting or emulsifying agents, pH buffering agents, and so forth.

In some embodiments, a composition comprising a compound described herein takes the form of a liquid where the agents are present in solution, in suspension or both. In some embodiments, when the composition is administered as a solution or suspension a first portion of the agent is present in solution and a second portion of the agent is present in particulate form, in suspension in a liquid matrix. In some embodiments, a liquid composition includes a gel formulation. In other embodiments, the liquid composition is aqueous.

Useful aqueous suspensions optionally contain one or more polymers as suspending agents. Useful polymers include water-soluble polymers such as cellulosic polymers, e.g., hydroxypropyl methylcellulose, and water-insoluble polymers such as cross-linked carboxyl-containing polymers. Useful compositions optionally comprise an mucoadhesive polymer, selected for example from carboxymethylcellulose, carbomer (acrylic acid polymer), poly(methylmethacrylate), polyacrylamide, polycarbophil, acrylic acid/butyl acrylate copolymer, sodium alginate and dextran.

Useful compositions optionally include solubilizing agents to aid in the solubility of a compound described herein. The term “solubilizing agent” generally includes agents that result in formation of a micellar solution or a true solution of the agent. Solubilizing agents include certain acceptable nonionic surfactants, for example polysorbate 80, and ophthalmically acceptable glycols, polyglycols, e.g., polyethylene glycol 400, and glycol ethers.

Useful compositions optionally include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

Useful compositions optionally include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

Certain useful compositions optionally include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

Some useful compositions optionally include one or more surfactants to enhance physical stability or for other purposes. Suitable nonionic surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40.

Certain useful compositions optionally one or more antioxidants to enhance chemical stability where required. Suitable antioxidants include, by way of example only, ascorbic acid and sodium metabisulfite.

In some embodiments, aqueous suspension compositions are packaged in single-dose non-reclosable containers. In alternative embodiments, multiple-dose reclosable containers are used, in which case it is typical to include a preservative in the composition.

In various embodiments, any delivery system for hydrophobic pharmaceutical compounds is employed. Liposomes and emulsions are examples of delivery vehicles or carriers for hydrophobic drugs. In some or any embodiments, certain organic solvents such as N-methylpyrrolidone are employed. In some embodiments, the compounds are delivered using a sustained-release system, such as semipermeable matrices of solid hydrophobic polymers containing the therapeutic agent. Various sustained-release materials are utilized in the embodiments herein. In some or any embodiments, sustained-release capsules release the compounds for a few weeks up to over 100 days. In some embodiments, depending on the chemical nature and the biological stability of the therapeutic reagent, additional strategies for protein stabilization are employed.

In some or any embodiments, the formulations or compositions described herein benefit from and/or optionally comprise antioxidants, metal chelating agents, thiol containing compounds and other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (l) pentosan polysulfate and other heparinoids, (m) divalent cations such as magnesium and zinc; or (n) combinations thereof.

### Methods of Dosing and Treatment Regimens

In certain embodiments, there are provided methods for reducing the storage of material that is normally degraded in the lysosome and can abnormally accumulate in individuals with lysosomal storage diseases with compounds of Formula (I), (Ia), and/or (Ib), or pharmaceutically acceptable salts thereof. In some embodiments, the compound or salt alters the biogenesis, function or dynamics of the endosomal or lysosomal systems in a way that reduces the abundance of the material abnormally stored in the lysosome in lysosomal storage diseases. In some embodiments, the compound or pharmaceutically acceptable salt thereof alters the biogenesis, functions, or dynamics of the endoplasmic reticulum or Golgi apparatus in a way that reduces the abundance of the material abnormally stored in the lysosome in lysosomal storage diseases. In some embodiments, the compound or pharmaceutically acceptable salt thereof alters the expression or function of cellular metabolic and catabolic pathways ways that reduce the abundance of the material abnormally stored in the lysosome in lysosomal storage diseases.

In some or any embodiments, the method can be conducted in living bodies of mammals. In such a case, the compounds may be administered to the mammals.

In some or any embodiments, the compounds described herein are used in the preparation or manufacture of medicaments for the treatment of lysosomal storage diseases or conditions. In some embodiments, a method for treating any of the diseases or conditions described herein involves administration to a subject in need of such treatment a pharmaceutical composition containing at least one compound described herein, or a pharmaceutically acceptable salt, pharmaceutically acceptable N-oxide, pharmaceutically active metabolite, pharmaceutically acceptable prodrug, or pharmaceutically acceptable solvate thereof, in a therapeutically effective amount to said subject.

In some or any embodiments, provided is a method of treating or ameliorating a medical condition, comprising administering to a subject in need thereof a compound according to any of the various embodiments described herein or a pharmaceutical composition according to any of the various embodiments described herein.

In some or any embodiments, provided herein is a method of treating or ameliorating a lysosomal storage disease comprising administering to a subject in need of treatment a therapeutically-effective amount of a compound of Formula (I), (Ia), and/or (Ib), optionally as a tautomer, a single stereoisomer or mixture of stereoisomers thereof and additionally optionally as a pharmaceutically acceptable salt thereof. In some or any embodiments, provided herein is a method of treating or ameliorating a lysosomal storage disease comprising administering to a subject in need of treatment a therapeutically-effective amount of a pharmaceutically acceptable composition comprising a compound of Formula (I), (Ia), and/or (Ib), optionally as a tautomer, a single stereoisomer or mixture of stereoisomers thereof and additionally optionally as a pharmaceutically acceptable salt thereof, and one or more excipients. In some or any embodiments, the lysosomal storage disease is selected from amyloid diseases (such as chronic hemodialysis-related amyloidosis) and an MPS disorder.

Lysosomal storage disorders include, but are not limited to, activator deficiency/GM2 gangliosidosis, alpha-mannosidosis, aspartylglucosaminuria, cholesteryl ester storage disease, chronic hemodialysis-related amyloidosis, chronic hexosaminidase A deficiency, cystinosis, Danon disease, Fabry disease, Farber disease, fucosidosis, galactosialidosis, Gaucher Disease (such as Type I, Type II, or Type III), GM1 gangliosidosis (such as infantile, late infantile, juvenile, adult, or chronic), Infantile Free Sialic Acid Storage Disease/ISSD, juvenile hexosaminidase A deficiency, Krabbe disease (such as infantile onset or late onset), lysosomal acid lipase deficiency (such as early onset or late onset), metachromatic leukodystrophy, MPS disorders, Mucolipidosis I/Sialidosis, Mucolipidosis IIIC, Mucolipidosis type IV, multiple sulfatase deficiency, Niemann-Pick Disease (such as Type A, Type B, or Type C), Neuronal Ceroid Lipofuscinoses, CLN6 disease (such as atypical late infantile, late onset variant, early juvenile forms), Batten-Spielmeyer-Vogt/Juvenile NCL/CLN3 disease, Finnish Variant Late Infantile CLN5, Jansky-Bielschowsky disease/Late infantile CLN2/TPP1 Disease, Kufs/Adult-onset NCL/CLN4 disease, Northern Epilepsy/variant late infantile CLN8, Santavuori-Haltia/Infantile CLN1/PPT disease, Beta-mannosidosis, Pompe disease/Glycogen storage disease type II, pycnodysostosis, Sandhoff disease/GM2 Gangliosidosis (such as adult onset, infantile, or juvenile forms), Schindler disease, Salla disease/Sialic Acid Storage Disease, Tay-Sachs/GM2 gangliosidosis, and Wolman disease. In some embodiments, the compound or salt of Formula (I), (Ia) and/or (Ib) can be used to treat a lysosomal storage disorder.

MPS disorders (mucopolysaccharidoses) are lysosomal storage diseases caused by the inability to produce specific enzymes, which in turn leads to an abnormal storage of mucopolysaccharides. MPS disorders include conditions such as Hurler syndrome (MPS I H), Scheie syndrome (MPS I S), Hurler-Scheie syndrome (MPS I H-S), Hunter syndrome (MPS II), Sanfilippo syndrome (e.g. Sanfilippo A (MPS III A), Sanfilippo B (MPS III B), Sanfilippo C (MPS III C), and Sanfilippo D (MPS III D)), Morquio syndrome (e.g. Morquio A (MPS IV A) and Morquio B (MPS IV B)), Maroteaux-Lamy syndrome (MPS VI), Sly syndrome (MPS VII), MPS IX (hyaluronidase deficiency), I-cell disease (ML II), and Pseudo-Hurler polydystrophy (ML III). In some embodiments, the compound or pharmaceutically acceptable salt of Formula (I), (Ia) and/or (Ib) can be used to treat an MPS disorder. In some embodiments, the compound or pharmaceutically acceptable salt of Formula (I), (Ia) and/or (Ib) can be used to treat an MPS disorder selected from: Hunter syndrome (MPS II), Sanfilippo A syndrome (MPS III A), and Sanfilippo B syndrome (MPS III B). In some embodiments, the compound or pharmaceutically acceptable salt of Formula (I), (Ia) and/or (Ib) can be used to treat Hunter syndrome (MPS II).

In any of the aforementioned embodiments are further embodiments that include single administrations of the effective amount of the compound, including further embodiments in which the compound is administered to the subject (i) once; (ii) multiple times over the span of one day; (iii) continually; or (iv) continuously.

In any of the aforementioned embodiments are further embodiments in which administration is enteral, parenteral, or both, and wherein:

(a) the effective amount of the compound is systemically administered to the subject;

(b) the effective amount of the compound is administered orally to the subject;

(c) the effective amount of the compound is intravenously administered to the subject;

(d) the effective amount of the compound administered by inhalation;

(e) the effective amount of the compound is administered by nasal administration;

(f) the effective amount of the compound is administered by injection to the subject;

(g) the effective amount of the compound is administered topically (dermal) to the subject;

(h) the effective amount of the compound is administered by ophthalmic administration; and/or

(i) the effective amount of the compound is administered rectally to the subject.

In some or any embodiments, the compositions containing the compound(s) described herein are administered for prophylactic and/or therapeutic treatments. In some or any therapeutic applications, the compositions are administered to a patient already suffering from a disease or condition, in an amount sufficient to cure or at least partially arrest the symptoms of the disease or condition. In some embodiments, amounts effective for this use will depend on the severity and course of the disease or condition, previous therapy, the patient's health status, weight, and response to the drugs, and the judgment of the treating physician. In certain instances, it is considered appropriate for the caregiver to determine such therapeutically effective amounts by routine experimentation (including, but not limited to, a dose escalation clinical trial).

In some or any prophylactic applications, compositions containing the compounds described herein are administered to a patient susceptible to or otherwise at risk of a particular disease, disorder or condition. In some embodiments, the amount administered is defined to be a “prophylactically effective amount or dose.” In some or any embodiments of this use, the precise amounts of compound administered depend on the patient's state of health, weight, and the like. In some embodiments, it is considered appropriate for the caregiver to determine such prophylactically effective amounts by routine experimentation (e.g., a dose escalation clinical trial). In some or any embodiments, when used in a patient, effective amounts for this use will depend on the severity and course of the disease, disorder or condition, previous therapy, the patient's health status and response to the drugs, and the judgment of the treating physician.

In some or any embodiments are further embodiments that include multiple administrations of the effective amount of the compound, including further embodiments wherein:

(i) the compound is administered in a single dose;

(ii) the time between multiple administrations is every 6 hours;

(iii) the compound is administered to the subject every 8 hours.

In further or alternative embodiments, the method includes a drug holiday, wherein the administration of the compound is temporarily suspended or the dose of the compound being administered is temporarily reduced; at the end of the drug holiday, dosing of the compound is resumed. In some embodiments, the length of the drug holiday varies from 2 days to 1 year.

In certain instances, a patient's condition does not improve or does not significantly improve following administration of a compound or composition described herein and, upon the doctor's discretion the administration of the compounds is optionally administered chronically, that is, for an extended period of time, including throughout the duration of the patient's life in order to ameliorate or otherwise control or limit the symptoms of the patient's disease or condition.

In certain cases wherein the patient's status does improve or does not substantially improve, upon the doctor's discretion the administration of the compounds are optionally given continuously; alternatively, the dose of drug being administered is optionally temporarily reduced or temporarily suspended for a certain length of time (i.e., a “drug holiday”). In some or any embodiments, the length of the drug holiday varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes a reduction from about 10% to about 100%, including, by way of example only, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In some or any embodiments, once improvement of the patient's conditions has occurred, a maintenance dose is administered if necessary. In some embodiments, the dosage, e.g., of the maintenance dose, or the frequency of administration, or both, are reduced, as a function of the symptoms, to a level at which the improved disease, disorder or condition is retained. In some or any embodiments, however, patients are optionally given intermittent treatment on a long-term basis upon any recurrence of symptoms.

In some or any embodiments, the amount of a given agent that corresponds to an effective amount varies depending upon factors such as the particular compound, disease or condition and its severity, the identity (e.g., weight) of the subject or host in need of treatment. In some embodiments, the effective amount is, nevertheless, determined according to the particular circumstances surrounding the case, including, e.g., the specific agent that is administered, the route of administration, the condition being treated, and the subject or host being treated. In some or any embodiments, however, doses employed for adult human treatment is in the range of about 0.02 to about 5000 mg per day, in a specific embodiment about 1 to about 1500 mg per day. In various embodiments, the desired dose is conveniently presented in a single dose or as divided doses administered simultaneously (or over a short period of time) or at appropriate intervals, for example as two, three, four or more sub-doses per day.

In some or any embodiments, the pharmaceutical compositions described herein are in a unit dosage form suitable for single administration of precise dosages. In some instances, in unit dosage form, the formulation is divided into unit doses containing appropriate quantities of one or more compound. In some or any embodiments, the unit dosage is in the form of a package containing discrete quantities of the formulation. Non-limiting examples are packaged tablets or capsules, and powders in vials or ampoules. In some embodiments, aqueous suspension compositions are packaged in single-dose non-reclosable containers. In alternative embodiments, multiple-dose reclosable containers are used, in which case it is typical to include a preservative in the composition. By way of example only, formulations for parenteral injection are, in some embodiments, presented in unit dosage form, which include, but are not limited to ampoules, or in multi-dose containers, with an added preservative.

In some or any embodiments, the daily dosages appropriate for the compounds described herein described herein are from about 0.01 to about 2.5 mg/kg per body weight. In some embodiments, an indicated daily dosage in the larger subject, including, but not limited to, humans, is in the range from about 0.5 mg to about 100 mg, conveniently administered in divided doses, including, but not limited to, up to four times a day or in extended release form. In some or any embodiments, suitable unit dosage forms for oral administration comprise from about 1 to about 50 mg active ingredient. The foregoing ranges are merely suggestive, as the number of variables in regard to an individual treatment regime is large, and considerable excursions from these recommended values are not uncommon. In some or any embodiments, the dosages are altered depending on a number of variables, not limited to the activity of the compound used, the disease or condition to be treated, the mode of administration, the requirements of the individual subject, the severity of the disease or condition being treated, and the judgment of the practitioner.

In some or any embodiments, toxicity and therapeutic efficacy of such therapeutic regimens are determined by standard pharmaceutical procedures in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index and it can be expressed as the ratio between LD₅₀ and ED₅₀. In some or any embodiments, compounds exhibiting high therapeutic indices are preferred. In some embodiments, the data obtained from cell culture assays and animal studies is used in formulating a range of dosage for use in human. In specific embodiments, the dosage of such compounds lies within a range of circulating concentrations that include the ED₅₀ with minimal toxicity. In some or any embodiments, the dosage varies within this range depending upon the dosage form employed and the route of administration utilized.

### Kits/Articles of Manufacture

Articles of manufacture, comprising packaging material, a compound provided herein that is effective for treatment, prevention or amelioration of one or more symptoms of a lysosomal storage disease or condition, within the packaging material, and a label that indicates that the compound or composition, or pharmaceutically acceptable salt, pharmaceutically acceptable N-oxide, pharmaceutically active metabolite, pharmaceutically acceptable prodrug, or pharmaceutically acceptable solvate thereof, is used for modulating lysosomal storage, or for treatment, prevention or amelioration of one or more symptoms of disease or condition in need of modulation of lysosomal storage, are provided.

For use in the therapeutic applications described herein, kits and articles of manufacture are also described herein. In various embodiments, such kits comprise a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In some embodiments, the containers are formed from a variety of materials such as glass or plastic.

In some embodiments, the articles of manufacture provided herein contain packaging materials. Packaging materials for use in packaging pharmaceutical products include, but are not limited to, blister packs, bottles, tubes, inhalers, pumps, bags, vials, containers, syringes, bottles, and any packaging material suitable for a selected formulation and intended mode of administration and treatment.

In some embodiments, the container(s) described herein comprise one or more compounds described herein, optionally in a composition or in combination with another agent as disclosed herein. The container(s) optionally have a sterile access port (for example in some embodiments the container is an intravenous solution bag or a vial having a stopper pierceable by a hypodermic injection needle). Such kits optionally comprise a compound with an identifying description or label or instructions relating to its use in the methods described herein.

In some embodiments, a kit will comprises one or more additional containers, each with one or more of various materials (such as reagents, optionally in concentrated form, and/or devices) desirable from a commercial and user standpoint for use of a compound described herein. Non-limiting examples of such materials include, but are not limited to, buffers, diluents, filters, needles, syringes; carrier, package, container, vial and/or tube labels listing contents and/or instructions for use, and package inserts with instructions for use. A set of instructions is optionally included.

In some or any embodiments, a label is on or associated with the container. In some embodiments, a label is on a container when letters, numbers or other characters forming the label are attached, molded or etched into the container itself; a label is associated with a container when it is present within a receptacle or carrier that also holds the container, e.g., as a package insert. In some or any embodiments, a label indicates that the contents are to be used for a specific therapeutic application. In some embodiments, the label indicates directions for use of the contents, such as in the methods described herein.

In some or any embodiments, the pharmaceutical compositions are presented in a pack or dispenser device which contains one or more unit dosage forms containing a compound provided herein. In some embodiments, the pack contains a metal or plastic foil, such as a blister pack. The pack or dispenser device is optionally accompanied by instructions for administration. In some embodiments, the pack or dispenser is accompanied with a notice associated with the container in form prescribed by a governmental agency regulating the manufacture, use, or sale of pharmaceuticals, which notice is reflective of approval by the agency of the form of the drug for human or veterinary administration. In some or any embodiments, such notice is, for example, the labeling approved by the U.S. Food and Drug Administration for prescription drugs, or the approved product insert. In some embodiments, compositions containing a compound provided herein are formulated in a compatible pharmaceutical carrier and are placed in an appropriate container labeled for treatment of an indicated condition.

Enzyme replacement therapy means administering an enzyme which is missing or deficient in a patient with a lysosomal storage disorder (e.g. MPS II).

Any combination of the groups described above for the various variables is contemplated herein.

### Preparation of Compounds

The following are illustrative examples of how the compounds can be prepared and tested. Although the examples can represent only some embodiments, it should be understood that the following examples are illustrative and not limiting.

In a further aspect, it is provided a method of making a compound, comprising synthesizing a compound as any of the various embodiments described above or below.

Compounds disclosed herein are commercially available or can be readily prepared from commercially available starting materials according to established methodology in the art of organic synthesis. General methods of synthesizing the compound can be found in, e.g., Stuart Warren and Paul Wyatt, Workbook for Organic Synthesis: The Disconnection Approach, second Edition, Wiley, 2010. Syntheses of the compounds are described in detail in U.S. Pat. Nos. 6,858,606; 7,745,436; 7,851,466; and 7,863,270; said syntheses are incorporated herein by reference.

In some embodiments, individual stereoisomers of compounds are prepared synthetically from commercially available starting materials which contain asymmetric or chiral centers or by preparation of racemic mixtures followed by resolution. These methods of resolution are exemplified by (1) attachment of a mixture of enantiomers to a chiral axillary, separation of the resulting mixture of diastereomers by recrystallization or chromatography and liberation of the optically pure product from the auxiliary or (2) direct separation of the mixture of optical enantiomers on chiral chromatographic column.

Materials were obtained from commercial suppliers and were used without further purification. Air or moisture sensitive reactions were conducted under argon atmosphere using oven-dried glassware and standard syringe/septa techniques. ¹H NMR spectra were measured at 400 MHz unless stated otherwise and data were reported as follows in ppm (δ) from the internal standard (TMS, 0.0 ppm): chemical shift (multiplicity, integration, coupling constant in Hz).

### Biological Examples

The following describes ways in which the compounds described herein were tested to measure in vitro activity in cell-based assays. A person of ordinary skill in the art would know that variations in the assay conditions could be used to determine the activity of the compounds.

### MPS II Cellular Assay

Dermal fibroblasts from humans with an MPS disorder and from healthy donors were grown. Cells were expanded and kept in culture for analysis.

Human foreskin fibroblasts are obtained from the American Type Culture Collection (CRL-1634). Human dermal fibroblasts derived from biopsies of individuals are purchased from Coriell Institute: clinically healthy individuals, GM00408, GM00409, GM00200 (clinically unaffected sibling of an individual with metachromatic leukodystrophy), GM05659, GM08398, and GM15871 (clinically unaffected sibling of an individual with unclassified Ehlers-Danlos syndrome); heterozygous MPS I carriers, GM01392, GM01393 and GM0003; homozygous individuals affected with MPS I, GM06214, GM11495, GM00034, GM01391 and GM01256; individuals with MPS II, GM01896, GM03181, GM00615 and GM00298; individuals with MPS IIIA, GM00879, GM00643, GM00934, GM06110 and GM00629; MPS IIIB, GM01426; MPS IIIC, GM05157; MPS IIID, GM05093; MPS VI, GM00519; and MPS VII, GM00121. All cells are grown in DMEM containing 50 U ml⁻¹ penicillin, 50 μg ml⁻¹ streptomycin, 2 mM glutamine and 10% FBS. Cells are seeded on 15-cm-diameter tissue culture dishes, grown to confluence and maintained in culture for analysis. Sulfamidase activity in cell extracts is measured with 4-methylumbelliferyl-α-D-N-sulfoglucosaminide according the vendors instructions substituting Tris-acetate buffer (pH 6.5; Moscerdam).

Cells are treated with a compound disclosed herein at a concentration at or below 30 μM for 4 to 21 days.

Cell monolayers are washed with phosphate buffered saline and detached by treatment with GIBCO trypsin-EDTA solution (Invitrogen). After centrifugation, the supernatant is removed and the cells are lysed in 0.5 ml of 0.1 N sodium hydroxide. The amount of protein is determined by bicinchoninic acid (BCA assay, Thermo Scientific). GAGs are isolated by anion exchange chromatography and digested with glycosaminoglycan lyases (IBEX).

Enzymatically depolymerized GAG preparations are differentially mass labeled by reductive amination with [¹²C₆]aniline. Briefly, heparan sulfate disaccharides (1-10 pmol) are dried down in a centrifugal evaporator and reacted with [¹²C₆]aniline or [¹³C₆]aniline (15 μL, 165 μmol) and 15 μL of 1 M NaCNBH₃ (Sigma-Aldrich) freshly prepared in dimethylsulfoxide/acetic acid (7:3, v/v) is added to each sample. Reactions are carried out at 37° C. for 16 h and then dried in a centrifugal evaporator. Unsubstituted amines are reacted with propionic anhydride (Sigma-Aldrich). Dried samples are reconstituted in 20 μL of 50% methanol, and 3 μL of propionic anhydride (Sigma-Aldrich, 23.3 μmol) is added. Reactions are carried out at −23° C. for 2 h. Acylated disaccharides were subsequently aniline tagged as described above. Each sample is mixed with commercially available standard unsaturated disaccharides (Seikagaku), standard N-sulfoglucosamine, glucosamine-6-sulfate, N-acetylgalactosamine-4-sulfate and N-acetylgalactosamine-6-sulfate (Sigma-Aldrich), and/or synthesized α-L-idopyranosyluronate-(1,4)-2-N-acetyl-2-deoxy-α/β-D-glucopyranoside (IOSO). All standards are tagged with [¹³C₆]aniline (Sigma-Aldrich). Samples are then analyzed by LC-MS using an LTQ Orbitrap Discovery electrospray ionization mass spectrometer (Thermo Scientific) equipped with quaternary HPLC pump (Finnigan Surveyor MS pump) and a reverse-phase capillary column.

For MPS II, GM01896 cells were treated with a compound described herein and the heparan sulfate disaccharide detected was [¹²C₆]aniline-tagged I2a4, I2a6, I2S0 and/or I2S6. Extracted ion chromatograms were obtained. Cell viability was monitored using alamar blue or ATP quantification. IC₅₀'s were calculated using curve fitting within the ChemInnovation CBIS database system.

The MPS II cellular assay can be adapted for use in testing cellular activity of compounds for treating MPS I where the biomarker detected is I0a6, I0a4, I0a10, I0S0 and/or I0S6; MPS IIIA where the biomarker detected is S0, S0U0S6, S0U2A6, S0U2S0, S0U2S6, S6U0A6, S6U0S0, S6U0S6, S6U2A6, S6U2S0, and/or S6U2S6; MPSIIIB where the biomarker detected is A0U0A6, A0U0S6, A0U2A0, A0U2S0, and/or A0U2S6; MPS IIIC where the biomarker detected is H0U0S6, H0U2A6, H0U2S0, H0U2S6, H6U0A6, H6U0S0, H6U0S6, H6U2A6, H6U2S0, and/or H6U2S6; MPS IIID where the biomarker detected is H6; MPS VI where the biomarker detected is a4; or MPS VII where the biomarker detected is G0a0, G0a4, G0a10, G0a6, G0A0, G0A6, G0S0, and/or G0S6. The biomarker code used in the above description has been detailed in: Lawrence, R. et al. Nature Methods 2008, 5(4), pages 291-292, which is incorporated herein by reference in its entirety.

Compound 11 was tested in the MPS II cellular assay and showed an IC₅₀=170 nM.

### MPS IIIA Disease Mouse Model

MPS IIIA mice (Sgsh−/−) are obtained from Jackson Laboratory (B6.Cg-Sgsh) and were housed in Association for Assessment and Accreditation of Laboratory Animal Care (AAALAC)-approved vivaria in the School of Medicine, University of California, San Diego, following standards and procedures approved by the local Institutional Animal Care and Use Committee for the ethical use of animals in experiments. Canine samples are provided by P. Dickson (University of California, Los Angeles—Harbor). MPS IIIA mice (see Bhaumik et al. Glycobiol 1999, 9, 1389) are injected with an amount (e.g., about 100 mg/kg/day) of a compound or pharmaceutically acceptable salt of the disclosure. Samples of mouse urine are incubated for one hour at 37° C. with two volumes of 0.1% cetylpyridinium chloride in 0.054 M sodium citrate (pH 4.8). Samples are centrifuged for 10 minutes at 3000 rpm and pellets are resuspended in 150 μL 2 M LiCl. Following addition of 800 μL absolute ethanol, samples are incubated at −20° C. for one hour and then centrifuged for 10 minutes at 3000 rpm. Pellets are resuspended in 200 μL of water, lyophilised, and then resuspended in 20 μL water. Purified glycosaminoglycan samples (0.2 μmol creatinine equivalents) are analysed on 40-50% linear gradient polyacrylamide gels. Samples are compared to results for a control MPS IIIA mouse (or population thereof) that was not exposed to the compound or salt of the disclosure.

### MPS IIIB Disease Mouse Model

Samples from MPS IIIB mice are provided by E. Neufeld (University of California, Los Angeles). Human urine samples without personal identifying information are obtained from donors from an MPS patient advocacy group with informed consent.

### Substrate Reduction Therapy

The non-specific inhibitor of sulfation, sodium chlorate, is used herein for validation or SRT. 30-60 mM sodium chlorate inhibits the synthesis of PAPs, the sulfate donor used in all cellular sulfation reactions including heparan sulfate biosynthesis. Cells grown in the presence of sodium chlorate produce heparan sulfate with reduced sulfation. In certain instances, in vitro MPS models used herein are based on measuring the accumulation of GAG fragments in cultured primary human fibroblast from MPS patients. In some instances, the GAGs that accumulate in MPS patients are much smaller than normal tissue GAGs and they lack a core protein on their reducing termini. Based on these features the in vitro MPS model used in certain instances herein is based on a method of tagging reducing ends of the GAGs with a detectable label and analyzing (i.e., detecting and/or measuring) the detectable labels using a device suitable for detecting the label (e.g., an HPLC with a fluorimeter).

Other objects, features and advantages of the compounds, methods and compositions described herein will become apparent from the following description. It should be understood, however, that the description and the specific examples, while indicating specific embodiments, are given by way of illustration only, since various changes and modifications within the spirit and scope of the present description will become apparent from this detailed description.

All publications including patents, patent applications and published patent applications cited herein are hereby incorporated by reference for all purposes.

1. A method for treating a lysosomal storage disorder in a subject in need thereof, the method comprising administering to the subject a compound, or a pharmaceutically acceptable salt thereof, according to Formula (I): 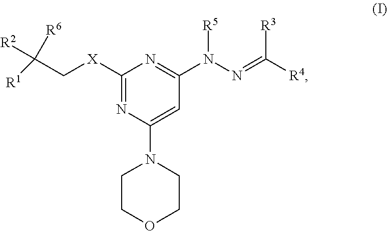
wherein
X is selected from the group consisting of: O, S, NH, and CH₂;
R¹ is selected from the group consisting of: C₁₋₃alkyl, —OR, oxazolidinonyl, morpholinyl, phenyl, pyridonyl, and pyridyl, wherein the C₁₋₃alkyl or phenyl is optionally substituted with 1-2 substituents independently selected from the group consisting of: —OCH₃ and —OH;
R is selected from H, C₁₋₃alkyl, phenyl, and pyridyl;
R² is selected from the group consisting of: H and CH₃; or
R¹ and R² taken together form a heterocyclic ring of 5-6 atoms optionally substituted with 1-2 C₁₋₃alkyl;
R³ is selected from the group consisting of: H and C₁₋₃alkyl;
R⁴ is selected from the group consisting of: aryl and heteroaryl, each of which is optionally substituted with C₁₋₃alkyl, halo, (C₁₋₃alkyl)-OH, —C(O)O(C₁₋₃alkyl), or —C(O)NH(C₁₋₃alkyl); or
R³ and R⁴ taken together form a carbocyclic, aryl, or heteroaryl ring, each of which is optionally substituted with 1-2 substituents independently selected from the group consisting of: halo, oxo, —OH, C₁₋₄alkyl, —O(C₁₋₄alkyl), and —NO₂;
R⁵ is selected from the group consisting of: H and CH₃;
R⁶ is selected from the group consisting of: H and CH₃; or
R² and R⁶ taken together form (═O) provided that R¹ and R² are not a heterocyclic ring.

2. The method of claim 1, wherein X is selected from the group consisting of: O and CH₂.

3. The method of claim 2, wherein X is O.

4. The method of any one of claims 1 to 3, wherein R¹ is selected from the group consisting of: oxazolidinonyl, morpholinyl, pyridonyl, pyridyl, and —O-pyridyl.

5. The method of claim 4, wherein R¹ is selected from the group consisting of: morpholinyl and pyridyl.

6. The method of claim 5, wherein R¹ is 2-pyridyl.

7. The method of any one of claims 1 to 6, wherein R² is H.

8. The method of any one of claims 1 to 7, wherein R³ is H.

9. The method of any one of claims 1 to 8, wherein R⁴ is a heteroaryl optionally substituted with C₁₋₃alkyl.

10. The method of claim 9, wherein R⁴ is indolyl.

11. The method of any one of claims 1 to 8, wherein R⁴ is an aryl optionally substituted with C₁₋₃alkyl, halo, (C₁₋₃alkyl)-OH, —C(O)O(C₁₋₃alkyl), or —C(O)NH(C₁₋₃alkyl).

12. The method of claim 11, wherein R⁴ is a phenyl optionally substituted with C₁₋₃alkyl, halo, (C₁₋₃alkyl)-OH, —C(O)O(C₁₋₃alkyl), or —C(O)NH(C₁₋₃alkyl).

13. The method of claim 12, wherein R⁴ is 3-methylphenyl.

14. The method of any one of claims 1 to 8, wherein R³ and R⁴ taken together form a heteroaryl optionally substituted with 1-2 substituents independently selected from the group consisting of: halo, oxo, —OH, C₁₋₄alkyl, and —O(C₁₋₄alkyl).

15. The method of any one of claims 1 to 8, wherein R³ and R⁴ taken together form an aryl optionally substituted with 1-2 substituents independently selected from the group consisting of: halo, —OH, C₁₋₄alkyl, —O(C₁₋₄alkyl), and —NO₂.

16. The method of any one of claims 1 to 15, wherein R⁵ is H.

17. The method of any one of claims 1 to 16, wherein R⁶ is H.

18. The method of any one of claims 1 to 6 and 8 to 15, wherein R², R⁵, and R⁶ are H.

19. The method of any one of claims 1 to 6 and 9 to 15, wherein R², R³, R⁵, and R⁶ are H.

20. A method for treating a lysosomal storage disorder in a subject in need thereof, the method comprising administering to the subject a compound, or a pharmaceutically acceptable salt thereof, according to Formula (Ia): 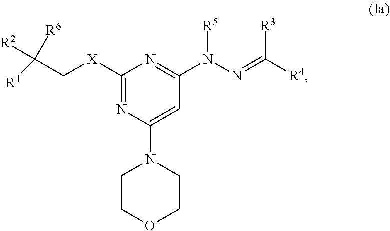
wherein
X is selected from the group consisting of: O, S, NH, and CH₂;
R¹ is selected from the group consisting of: C₁₋₃alkyl, —OR, morpholinyl, phenyl, pyridonyl, and pyridyl, wherein the C₁₋₃alkyl or phenyl is optionally substituted with 1-2 substituents independently selected from the group consisting of: —OCH₃ and —OH;
R is selected from H, C₁₋₃alkyl, phenyl, and pyridyl;
R² is selected from the group consisting of: H and CH₃; or
R¹ and R² taken together form a heterocyclic ring of 5-6 atoms optionally substituted with 1-2 C₁₋₃alkyl;
R³ is selected from the group consisting of: H and CH₃;
R⁴ is selected from the group consisting of: phenyl and indolyl, each of which is optionally substituted with C₁₋₃alkyl, halo, (C₁₋₃alkyl)-OH, —C(O)O(C₁₋₃alkyl), or —C(O)NH(C₁₋₃alkyl);
R⁵ is selected from the group consisting of: H and CH₃;
R⁶ is selected from the group consisting of: H and CH₃; or
R² and R⁶ taken together form (═O) provided that R¹ and R² are not a heterocyclic ring.

21. The method of claim 20, wherein X is selected from the group consisting of: O and CH₂.

22. The method of claim 21, wherein X is O.

23. The method of any one of claims 20 to 22, wherein R¹ is selected from the group consisting of: —O-pyridyl, morpholinyl, pyridonyl, and pyridyl.

24. The method of claim 23, wherein R¹ is 2-pyridyl.

25. The method of any one of claims 20 to 24, wherein R² is H.

26. The method of any one of claims 20 to 25, wherein R³ is H.

27. The method of any one of claims 20 to 26, wherein R⁴ is phenyl optionally substituted with C₁₋₃alkyl, halo, (C₁₋₃alkyl)-OH, —C(O)O(C₁₋₃alkyl), or —C(O)NH(C₁₋₃alkyl).

28. The method of claim 27, wherein R⁴ is 3-halophenyl.

29. The method of claim 27, wherein R⁴ is 3-methylphenyl.

30. The method of any one of claims 20 to 29, wherein R⁵ is H.

31. The method of any one of claims 20 to 30, wherein R⁶ is H.

32. The method of any one of claims 20 to 24 and 26 to 29, wherein R², R⁵, and R⁶ are H.

33. The method of any one of claims 20 to 24 and 27 to 29, wherein R², R³, R⁵, and R⁶ are H.

34. The method of claim 20, wherein the compound or pharmaceutically acceptable salt of Formula (Ia) is selected from the group consisting of:
(E)-4-(6-(2-((1H-indol-3-yl)methylene)hydrazinyl)-2-(3-(3,4-dimethoxyphenyl)-propyl)-pyrimidin-4-yl)morpholine;
(E)-4-(6-(2-((1H-indol-3-yl)methylene)hydrazinyl)-2-butoxypyrimidin-4-yl)morpholine;
(E)-4-(4-(2-((1H-indol-3-yl)methylene)hydrazinyl)-6-morpholinopyrimidin-2-yl)butan-1-ol;
(E)-4-(2-(2-(1,3-dioxan-2-yl)ethyl)-6-(2-((1H-indol-3-yl)methylene)hydrazinyl)pyrimidin-4-yl)morpholine;
(E)-4-(6-(2-((1H-indol-3-yl)methylene)hydrazinyl)-2-(3-methoxypropyl)pyrimidin-4-yl)morpholine;
(E)-3-((4-(2-((1H-indol-3-yl)methylene)hydrazinyl)-6-morpholinopyrimidin-2-yl)thio)propan-1-ol;
(E)-4-(6-(2-((1H-indol-3-yl)methylene)hydrazinyl)-2-((2,2-dimethyl-1,3-dioxolan-4-yl)methoxy)pyrimidin-4-yl)morpholine;
(E)-4-(6-(2-((1H-indol-3-yl)methylene)hydrazinyl)-2-(3,4-dimethoxyphenethoxy)pyrimidin-4-yl)morpholine;
(E)-4-(6-(2-((1H-indol-3-yl)methylene)hydrazinyl)-2-(2-(pyridin-2-yl)ethoxy)pyrimidin-4-yl)morpholine;
(E)-4-(6-(2-((1H-indol-3-yl)methylene)hydrazinyl)-2-(3-(pyridin-2-yl)propyl)pyrimidin-4-yl)morpholine;
(E)-4-(6-(2-(3-methylbenzylidene)hydrazinyl)-2-(2-(pyridin-2-yl)ethoxy)pyrimidin-4-yl)morpholine;
(E)-4-(6-(2-(3-ethylbenzylidene)hydrazinyl)-2-(2-(pyridin-2-yl)ethoxy)pyrimidin-4-yl)morpholine;
(E)-4-(6-(2-(3-methylbenzylidene)hydrazinyl)-2-(3-(pyridin-2-yl)propyl)pyrimidin-4-yl)morpholine;
(E)-4-(2-(2-(pyridin-2-yl)ethoxy)-6-(2-(1-(m-tolyl)ethylidene)hydrazinyl)pyrimidin-4-yl)morpholine;
(E)-4-(6-(2-((1H-indol-3-yl)methylene)-1-methylhydrazinyl)-2-(2-(pyridin-2-yl)ethoxy)pyrimidin-4-yl)morpholine;
(E)-4-(6-(2-((1H-indol-3-yl)methylene)hydrazinyl)-2-(2-(pyridin-3-yloxy)ethoxy)pyrimidin-4-yl)morpholine;
(E)-4-(6-(2-(3-methylbenzylidene)hydrazinyl)-2-(2-(pyridin-3-yloxy)ethoxy)pyrimidin-4-yl)morpholine;
(E)-4-(2-((1H-indol-3-yl)methylene)hydrazinyl)-N-butyl-6-morpholinopyrimidin-2-amine;
(E)-ethyl 3-(4-(2-(3-methylbenzylidene)hydrazinyl)-6-morpholinopyrimidin-2-yl)propanoate;
(E)-4-(6-(2-(3-methylbenzylidene)hydrazinyl)-2-(2-morpholinoethoxy)pyrimidin-4-yl)morpholine;
(E)-4-(6-(1-methyl-2-(3-methylbenzylidene)hydrazinyl)-2-(2-(pyridin-2-yl)ethoxy)pyrimidin-4-yl)morpholine;
(E)-4-(6-(1-methyl-2-((1-methyl-1H-indol-3-yl)methylene)hydrazinyl)-2-(2-(pyridin-2-yl)ethoxy)pyrimidin-4-yl)morpholine;
(E)-2-((4-(2-(3-methylbenzylidene)hydrazinyl)-6-morpholinopyrimidin-2-yl)oxy)ethanol;
(E)-3-((4-(2-(3-methylbenzylidene)hydrazinyl)-6-morpholinopyrimidin-2-yl)oxy)propan-1-ol;
(E)-4-(2-(2-methoxyethoxy)-6-(2-(3-methylbenzylidene)hydrazinyl)pyrimidin-4-yl)morpholine;
(E)-1-(2-((4-(2-(3-methylbenzylidene)hydrazinyl)-6-morpholinopyrimidin-2-yl)oxy)ethyl)pyridin-2(1H)-one;
(E)-4-(6-(2-(3-iodobenzylidene)hydrazinyl)-2-(2-(pyridin-2-yl)ethoxy)pyrimidin-4-yl)morpholine;
(E)-4-(6-(2-(3-fluorobenzylidene)hydrazinyl)-2-(2-(pyridin-2-yl)ethoxy)pyrimidin-4-yl)morpholine;
(E)-4-(6-(2-(3-fluorobenzylidene)hydrazinyl)-2-(2-(pyridin-2-yl)ethoxy)pyrimidin-4-yl)morpholine;
(E)-4-(6-(2-(3-bromobenzylidene)hydrazinyl)-2-(2-(pyridin-2-yl)ethoxy)pyrimidin-4-yl)morpholine;
(E)-methyl 3-((2-(6-morpholino-2-(2-(pyridin-2-yl)ethoxy)pyrimidin-4-yl)hydrazono)methyl)benzoate;
(E)-1-(2-((4-(2-(3-iodobenzylidene)hydrazinyl)-6-morpholinopyrimidin-2-yl)oxy)ethyl)pyridin-2(1H)-one;
(E)-N-methyl-3-((2-(6-morpholino-2-(2-(pyridin-2-yl)ethoxy)pyrimidin-4-yl)hydrazono)methyl)benzamide; and
(E)-(3-((2-(6-morpholino-2-(2-(pyridin-2-yl)ethoxy)pyrimidin-4-yl)hydrazono)methyl)phenyl)methanol.

35. A method for treating a lysosomal storage disorder in a subject in need thereof, the method comprising administering to the subject a compound, or a pharmaceutically acceptable salt thereof, according to Formula (Ib): 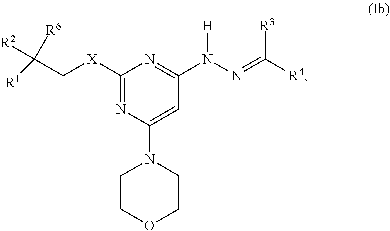
wherein
X is selected from the group consisting of: O and CH₂;
R¹ is selected from the group consisting of: C₁₋₃alkyl, oxazolidinonyl, and morpholinyl, and pyridyl, wherein the C₁₋₃alkyl is optionally substituted with 1-2 substituents independently selected from the group consisting of: —OCH₃ and —OH;
R² is selected from the group consisting of: H and CH₃;
R³ and R⁴ taken together form a carbocyclic, aryl, or heteroaryl ring, each of which is optionally substituted with 1-2 substituents independently selected from the group consisting of: halo, oxo, —OH, C₁₋₄alkyl, —O(C₁₋₄alkyl), and —NO₂;
R⁶ is selected from the group consisting of: H and CH₃.

36. The method of claim 35, wherein X is O.

37. The method of claim 35 or 36, wherein R¹ is morpholinyl.

38. The method of any one of claims 35 to 37, wherein R² is H.

39. The method of any one of claims 35 to 38, wherein R³ and R⁴ taken together form an aryl ring, optionally substituted with 1-2 substituents independently selected from the group consisting of: halo, oxo, —OH, C₁₋₄alkyl, —O(C₁₋₄alkyl), and —NO₂.

40. The method of any one of claims 35 to 38, wherein R³ and R⁴ taken together form a heteroaryl ring, optionally substituted with 1-2 substituents independently selected from the group consisting of: halo, oxo, —OH, and —O(C₁₋₄alkyl).

41. The method of any one of claims 35 to 38, wherein R³ and R⁴ taken together form a carbocyclic ring, optionally substituted with C₁₋₄alkyl.

42. The method of any one of claims 35 to 41, wherein R⁶ is H.

43. The method of any one of claims 35 to 37 and 39 to 41, wherein R² and R⁶ are H.

44. The method of claim 35, wherein the compound or pharmaceutically acceptable salt of Formula (Ib) is selected from the group consisting of:
(Z)-3-(2-(6-morpholino-2-(2-morpholinoethoxy)pyrimidin-4-yl)hydrazono)indolin-2-one;
(E)-4-(6-(2-(6-methylchroman-4-ylidene)hydrazinyl)-2-(2-morpholinoethoxy)pyrimidin-4-yl)morpholine;
(E)-4-(6-(2-(6-methyl-2,3-dihydro-1H-inden-1-ylidene)hydrazinyl)-2-(2-morpholinoethoxy)pyrimidin-4-yl)morpholine;
(E)-4-(6-(2-(2,3-dihydro-1H-inden-1-ylidene)hydrazinyl)-2-(2-morpholinoethoxy)pyrimidin-4-yl)morpholine;
(Z)-4-(6-(2-(benzofuran-3(2H)-ylidene)hydrazinyl)-2-(2-morpholinoethoxy)pyrimidin-4-yl)morpholine;
(E)-4-(6-(2-(3-methyl-2,3-dihydro-1H-inden-1-ylidene)hydrazinyl)-2-(2-morpholinoethoxy)pyrimidin-4-yl)morpholine;
(E)-4-(6-(2-(4-methyl-2,3-dihydro-1H-inden-1-ylidene)hydrazinyl)-2-(2-morpholinoethoxy)pyrimidin-4-yl)morpholine;
(E)-4-(6-(2-(5-methoxy-2,3-dihydro-1H-inden-1-ylidene)hydrazinyl)-2-(2-morpholinoethoxy)pyrimidin-4-yl)morpholine;
(E)-4-(6-(2-(6-methoxy-2,3-dihydro-1H-inden-1-ylidene)hydrazinyl)-2-(2-morpholinoethoxy)pyrimidin-4-yl)morpholine;
(E)-4-(6-(2-(2,3-dihydro-1H-inden-1-ylidene)hydrazinyl)-2-(2-morpholinoethoxy)pyrimidin-4-yl)morpholine;
(E)-4-(6-(2-(3,4-dihydronaphthalen-1(2H)-ylidene)hydrazinyl)-2-(2-morpholinoethoxy)pyrimidin-4-yl)morpholine;
(E)-4-(6-(2-(chroman-4-ylidene)hydrazinyl)-2-(2-morpholinoethoxy)pyrimidin-4-yl)morpholine;
(E)-4-(6-(2-(6-methoxy-3,4-dihydronaphthalen-1(2H)-ylidene)hydrazinyl)-2-(2-morpholinoethoxy)pyrimidin-4-yl)morpholine;
(E)-4-(6-(2-(7-methoxy-3,4-dihydronaphthalen-1(2H)-ylidene)hydrazinyl)-2-(2-morpholinoethoxy)pyrimidin-4-yl)morpholine;
(E)-4-(2-((4-morpholino-6-(2-(7-nitro-3,4-dihydronaphthalen-1(2H)-ylidene)hydrazinyl)pyrimidin-2-yl)oxy)ethyl)morpholine;
(E)-5-(2-(6-morpholino-2-(2-morpholinoethoxy)pyrimidin-4-yl)hydrazono)-5,6,7,8-tetrahydronaphthalen-2-ol;
(E)-4-(6-(2-(5,7-dimethyl-3,4-dihydronaphthalen-1(2H)-ylidene)hydrazinyl)-2-(2-morpholinoethoxy)pyrimidin-4-yl)morpholine;
(E)-4-(6-(2-(6,7-dimethoxy-3,4-dihydronaphthalen-1(2H)-ylidene)hydrazinyl)-2-(2-morpholinoethoxy)pyrimidin-4-yl)morpholine;
(E)-4-(6-(2-(4-methyl-3,4-dihydronaphthalen-1(2H)-ylidene)hydrazinyl)-2-(2-morpholinoethoxy)pyrimidin-4-yl)morpholine;
(Z)-1-methyl-3-(2-(6-morpholino-2-(2-morpholinoethoxy)pyrimidin-4-yl)hydrazono)indolin-2-one;
(E)-3-(2-((4-(2-(6-methyl-2,3-dihydro-1H-inden-1-ylidene)hydrazinyl)-6-morpholinopyrimidin-2-yl)oxy)ethyl)oxazolidin-2-one;
(E)-3-(2-((4-(2-(6-hydroxy-3,4-dihydronaphthalen-1(2H)-ylidene)hydrazinyl)-6-morpholinopyrimidin-2-yl)oxy)ethyl)oxazolidin-2-one;
(E)-2-methyl-4-(4-(2-(6-methyl-2,3-dihydro-1H-inden-1-ylidene)hydrazinyl)-6-morpholinopyrimidin-2-yl)butan-2-ol;
(E)-5-(2-(2-(3-hydroxy-3-methylbutyl)-6-morpholinopyrimidin-4-yl)hydrazono)-5,6,7,8-tetrahydronaphthalen-2-ol;
(E)-1-(2-(6-morpholino-2-(2-morpholinoethoxy)pyrimidin-4-yl)hydrazono)-2,3-dihydro-1H-inden-4-ol;
(E)-1-(2-(6-morpholino-2-(2-morpholinoethoxy)pyrimidin-4-yl)hydrazono)-2,3-dihydro-1H-inden-5-ol;
(Z)-3-(2-(6-morpholino-2-(2-morpholinoethoxy)pyrimidin-4-yl)hydrazono)-2,3-dihydrobenzofuran-6-ol;
(E)-5-(2-(6-morpholino-2-(2-morpholinoethoxy)pyrimidin-4-yl)hydrazono)-5,6,7,8-tetrahydronaphthalen-1-ol;
(E)-4-(6-(2-(6-fluorochroman-4-ylidene)hydrazinyl)-2-(2-morpholinoethoxy)pyrimidin-4-yl)morpholine;
(E)-4-(6-(2-(5-fluoro-2,3-dihydro-1H-inden-1-ylidene)hydrazinyl)-2-(2-morpholinoethoxy)pyrimidin-4-yl)morpholine;
(E)-4-(2-(6-morpholino-2-(2-morpholinoethoxy)pyrimidin-4-yl)hydrazono)-4,5,6,7-tetrahydrobenzo[c][1,2,5]oxadiazole;
4-(2-((4-morpholino-6-((E)-2-((4aR,8aS)-octahydronaphthalen-1(2H)-ylidene)hydrazinyl)pyrimidin-2-yl)oxy)ethyl)morpholine;
4-(6-(2-(4-(tert-butyl)cyclohexylidene)hydrazinyl)-2-(2-morpholinoethoxy)pyrimidin-4-yl)morpholine;
(E)-4-(6-(2-(2-methylcyclohexylidene)hydrazinyl)-2-(2-morpholinoethoxy)pyrimidin-4-yl)morpholine;
4-(6-(2-cyclopentylidenehydrazinyl)-2-(2-morpholinoethoxy)pyrimidin-4-yl)morpholine
(E)-4-(6-(2-(bicyclo[2.2.1]heptan-2-ylidene)hydrazinyl)-2-(2-morpholinoethoxy)pyrimidin-4-yl)morpholine;
(E)-4-(6-(2-(6-chlorothiochroman-4-ylidene)hydrazinyl)-2-(2-morpholinoethoxy)pyrimidin-4-yl)morpholine;
(E)-6-chloro-4-(2-(6-morpholino-2-(2-morpholinoethoxy)pyrimidin-4-yl)hydrazono)thiochroman 1,1-dioxide;
(E)-4-(6-(2-(6-methyl-4H-chromen-4-ylidene)hydrazinyl)-2-(2-morpholinoethoxy)pyrimidin-4-yl)morpholine;
(E)-4-(6-(2-(6-chloro-4H-chromen-4-ylidene)hydrazinyl)-2-(2-morpholinoethoxy)pyrimidin-4-yl)morpholine; and
(E)-4-(6-(2-(6,7-dihydrobenzofuran-4(5H)-ylidene)hydrazinyl)-2-(2-morpholinoethoxy)pyrimidin-4-yl)morpholine.

45. A method for treating a lysosomal storage disorder in a subject in need thereof, the method comprising administering to the subject a compound, or a pharmaceutically acceptable salt thereof, having the structure:

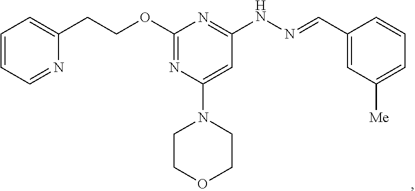 [SMILES: Cc1cccc(/C=N/Nc2cc(N3CCOCC3)nc(OCCc3ccccn3)n2)c1 ; ","]

46. The method of any one of claims 1-45, wherein the lysosomal storage disorder is selected from the group consisting of: activator deficiency/GM2 gangliosidosis, alpha-mannosidosis, aspartylglucosaminuria, cholesteryl ester storage disease, chronic hemodialysis-related amyloidosis, chronic hexosaminidase A deficiency, cystinosis, Danon disease, Fabry disease, Farber disease, fucosidosis, galactosialidosis, Gaucher Disease, GM1 gangliosidosis, Infantile Free Sialic Acid Storage Disease/ISSD, juvenile hexosaminidase A deficiency, Krabbe disease, lysosomal acid lipase deficiency, metachromatic leukodystrophy, MPS disorders, Mucolipidosis I/Sialidosis, Mucolipidosis IIIC, Mucolipidosis type IV, multiple sulfatase deficiency, Niemann-Pick Disease, Neuronal Ceroid Lipofuscinoses, CLN6 disease, Batten-Spielmeyer-Vogt/Juvenile NCL/CLN3 disease, Finnish Variant Late Infantile CLN5, Jansky-Bielschowsky disease/Late infantile CLN2/TPP1 Disease, Kufs/Adult-onset NCL/CLN4 disease, Northern Epilepsy/variant late infantile CLN8, Santavuori-Haltia/Infantile CLN1/PPT disease, Beta-mannosidosis, Pompe disease/Glycogen storage disease type II, pycnodysostosis, Sandhoff disease/GM2 Gangliosidosis, Schindler disease, Salla disease/Sialic Acid Storage Disease, Tay-Sachs/GM2 gangliosidosis, and Wolman disease.

47. The method of claim 46, wherein the lysosomal storage disorder is an MPS disorder selected from the group consisting of: Hurler syndrome, Scheie syndrome, Hurler-Scheie syndrome, Hunter syndrome, Sanfilippo syndrome, Morquio syndrome, Maroteaux-Lamy syndrome, Sly syndrome, MPS IX, I-cell disease, and Pseudo-Hurler polydystrophy.

48. The method of claim 47, wherein the MPS disorder is Hunter syndrome.

49. The method of any one of claims 1-48, wherein the pharmaceutically acceptable salt thereof is selected from the group consisting of: hydrochloric acid, nitric acid, sulfuric acid, hydrobromic acid, hydroiodic acid, perchloric acid, phosphoric acid, alkylsulfonic acids, aryl sulfonic acids, halogenated alkylsulfonic acids, halogenated acetic acids, picric acid, oxalic acid, citric acid, formic acid, ascorbic acid and benzoic acid.

50. The method of claim 49, wherein the pharmaceutically acceptable salt thereof is a methanesulfonic acid salt.

51. The method of claim 49, wherein the pharmaceutically acceptable salt thereof is a hydrobromic acid salt.

52. The method of claim 49, wherein the pharmaceutically acceptable salt thereof is a hydrochloric acid salt.

53. A method for treating Hunter syndrome in a subject in need thereof, the method comprising administering to the subject a compound, or a pharmaceutically acceptable salt thereof, having the structure:

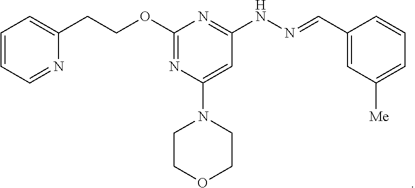 [SMILES: Cc1cccc(/C=N/Nc2cc(N3CCOCC3)nc(OCCc3ccccn3)n2)c1 ; "."]

54. The method of claim 53, wherein the pharmaceutically acceptable salt thereof is selected from the group consisting of: hydrochloric acid, nitric acid, sulfuric acid, hydrobromic acid, hydroiodic acid, perchloric acid, phosphoric acid, alkylsulfonic acids, aryl sulfonic acids, halogenated alkylsulfonic acids, halogenated acetic acids, picric acid, oxalic acid, citric acid, formic acid, ascorbic acid and benzoic acid.

55. The method of claim 54, wherein the pharmaceutically acceptable salt thereof is a methanesulfonic acid salt.

56. The method of claim 54, wherein the pharmaceutically acceptable salt thereof is a hydrobromic acid salt.

57. The method of claim 54, wherein the pharmaceutically acceptable salt thereof is a hydrochloric acid salt.
